(19) United States
(12) Patent Application Publication
(10) Pub. No.: US 2004/0253642 A1
(43) Pub. Date: Dec. 16, 2004

(54) System and method for multidimensional evaluation of combinations of compositions

(76) Inventors: Grant Zimmermann, Somerville, MA (US); Raymond A. Molnar, Boston, MA (US); Joseph Lehar, Lexington, MA (US); Jason Fong, Philadelphia, PA (US); Curtis T. Keith, Boston, MA (US); George Serbedzija, Sudbury, MA (US); Margaret S. Lee, Middleton, MA (US); Edward R. Jost-Price, Jamaica Plain, MA (US); Nicole Hurst, West Roxbury, MA (US); Alexis Borisy, Arlington, MA (US); Michael A. Foley, Chestnut Hill, MA (US); Brent Stockwell, Boston, MA (US)

Correspondence Address: BROMBERG & SUNSTEIN LLP, 125 SUMMER STREET, BOSTON, MA, 02110-1618, US

(21) Appl. No.: 10/863,592

(22) Filed: Jun. 7, 2004

Related U.S. Application Data
(60) Provisional application No. 60/476,342, filed on Jun. 6, 2003.

(51) Int. Cl. G01N 33/53

(52) U.S. Cl. 435/7.1

(57) ABSTRACT

Embodiments of the invention are directed toward methods and devices for constructing assay arrays including a combination of constituent compositions from constituent arrays. The combined compositions are evaluated, in some embodiments of the invention, to identify combinations with a combination effect. Other embodiments of the invention are directed toward constructing constituent arrays in various configurations to facilitate the production of assay arrays. Such embodiments include: constructing constituent arrays and assay arrays with corresponding composition and assay control sets; constructing constituent arrays with a unique set of origin locations and corresponding sets of derivative locations; varying the concentrations utilized for a constituent composition; and composing assay arrays corresponding to a virtual sparse assay array. Other embodiments of the invention are directed towards systems and method of evaluating the activity of combined compositions in an assay array.

### CROSS REFERENCE TO RELATED APPLICATIONS

The present application claims priority from a U.S. provisional patent application with Ser. No. 60/476,342 filed on Jun. 6, 2003. The present application is also related to a U.S. patent application with the same inventors and title as the present application, bearing attorney docket number 2729/104, and filed on the same day as the present application. These applications are all hereby incorporated herein by reference in their entirety.

### TECHNICAL FIELD

The present invention relates to systems and methods for evaluation of compositions, and in particular for multidimensional evaluation of combinations of compositions.

### BACKGROUND ART

Research on chemicals, drugs, and therapeutics rely upon laboratory testing of compositions to evaluate the suitability of a composition's contents to a specific application. In drug testing, discovery of unique combinations of substances that provide clinical efficacy may require the testing of a large number of combinations of candidate substances. In addition, the effective concentration of each substance in a specific combination may also require identification. In identifying what combinations of substances may be useful, each combination may need to be exposed to a large variety of test elements and conditions in order to determine the optimal activity of the combination. Exploration of such a large, multivariate space may be prohibitively costly in terms of time and resources if manual testing of all possible combinations is required.

High throughput screening may hasten the discovery process, and economize the use of resources, through the use of automated machinery to prepare the necessary samples for testing, thus facilitating testing and evaluation of the activity of a candidate composition. The screening process may aid identification of candidate compositions. Follow-on screens may further identify which candidates may be particularly effective, and what concentrations of the constituents of a combination may be optimal.

Even with the use of automated machinery, identification of useful combinations of compounds, from a large library of individual candidates, remains a time-consuming, costly task. Furthermore, testing errors may further hinder the process of candidate identification by providing false negative results, causing scientists to overlook viable candidates, and false positive results, causing scientists to spend scarce resources analyzing ultimately unattractive candidates. A need exists to provide methods and systems which may further enhance the speed and accuracy of testing a large number of compositions combined in a variety of mixtures.

### SUMMARY OF THE INVENTION

In an embodiment of the invention there is provided a method for evaluating the activity of a set of combined compositions which is formed from a common plurality of constituent compositions. The method includes the steps of providing for each constituent composition, a constituent array of locations each holding a specific concentration of a constituent composition, the number of the arrays corresponding to the plurality of constituent compositions; providing an assay array of locations, each location of the assay array corresponding to a member of the set and being associated with a designated aliquot from each of the constituent arrays, wherein each aliquot is one of zero and non-zero; and evaluating the activity of combined composition at each location of the assay array. Alternate embodiments of the invention include constituent compositions wherein one or more entities are approved by a governmental regulatory agency for administration to a patient; have an established safety profile, have a recognized pharmacological profile, or have a recognized toxicity profile. Combined compositions may also include an evaluative composition pertinent to evaluating the activity of the combined composition, the evaluative composition optionally including at least one test entity.

Another embodiment of the invention involves a method for evaluating the activity of a set of combined compositions which is formed from a common plurality of constituent compositions, wherein a particular concentration of at least one constituent composition in the assay array is designated based upon activity data of the at least one constituent composition, or corresponds approximately with a designated activity of the at least one constituent composition in the assay array. A related method includes evaluating an activity of the at least one constituent composition before providing its constituent array of locations, wherein the activity data is based upon the evaluated activity of the at least one constituent composition before providing its constituent array of locations. Alternatively, the activity data is based upon known activity data of the at least one constituent composition. The activity data may be represented in the form of at least one value of inhibition. As well, a plurality of particular concentrations of the at least one constituent composition in the assay array may be based upon the activity data of the at least one constituent composition. The plurality of particular concentrations may correspond approximately with designated values of activity, such as inhibitions, of the at least one constituent composition. In particular, the designated values of inhibition may be approximately between 20% and 80% of a maximum inhibition of the at least one constituent composition.

In another alternative, the plurality of particular concentrations may include at least one concentration corresponding approximately to a selected value of activity of the at least one constituent composition based upon the activity data of the at least one constituent composition, and at least one other particular concentration based upon the selected value of activity. In particular, the at least one other particular concentration may be based upon a product of the selected concentration and a predetermined multiplicative factor. For example, the selected value of activity may be a value of inhibition of 80% of a maximum inhibition of the at least one constituent composition, and the at least one specific concentration corresponds to approximately a two-fold multiple dilution from a concentration corresponding to the value of inhibition of 80% of the maximum inhibition of the at least one constituent composition.

In another embodiment of the invention, at least one constituent array includes a series of members having successively greater dilutions of such constituent composition. One embodiment includes successively greater dilutions that encompass a total range of a factor of at least approximately 50,000, achieved in steps of a factor of at least approximately 3. A second embodiment includes successively greater dilutions that encompass a total range of a factor of at least approximately 1,000, achieved in steps of a factor of at least approximately 4. A third embodiment includes successively greater dilutions that encompass a total range of a factor of at least approximately 250, achieved in steps of a factor of at least approximately 2.

Other embodiments may require each location of any constituent array to have at least one corresponding location in any of the other constituent arrays, and the designated aliquot from each of the constituent arrays be taken from corresponding locations of the constituent arrays; all arrays to have a common number of locations in corresponding positions of their respective physical objects; and each array being embodied in at least one plate, each location of each plate optionally realized by a well.

In an alternative embodiment of the invention, each constituent array includes at least one constituent composition with varying concentration in a plurality of locations, and wherein at least one concentration of the at least one constituent composition of one particular constituent array is not combined with every concentration of another constituent composition associated with another constituent array in the assay array.

Another alternate embodiment of the invention includes, for each constituent array of locations, providing an origin set of unique locations in each constituent array, each location associated with a quantity of constituent composition associated with such array; and providing, for each location of the origin set, a derivative set of unique locations in each constituent array, each location of a specific derivative set having a portion of constituent composition obtained from a location of the origin set. The origin set may be embodied on a single physical object. Additionally, each location of any constituent array may have a corresponding location in any of the other constituent arrays, and a plurality of locations from an origin set and its corresponding derivative set of a given constituent array may be distinct from any locations of such constituent array that correspond to locations of an origin set and its corresponding derivative set in any other constituent array. Each of a plurality of locations of a derivative set may include diluent.

In a particular alternate embodiment, constituent arrays have a geometrically similarly configured plurality of locations, arranged in rows and columns. The constituent arrays are oriented such that at least one array, a X constituent array, has an origin set of locations arranged in a vertical column with each derivative set of locations oriented as a horizontal row of locations adjacent to its corresponding origin location, and at least one array, a Y constituent array, has an origin set of locations arranged in a horizontal row with each derivative set of locations oriented as a vertical column of locations adjacent to its corresponding origin location. The location of the combined compositions of the X and Y constituent arrays into an assay array preserves the relative orientation of the constituent compositions of the constituent arrays. Alternatively, each of a first and a second constituent array may have an identically configured predetermined number of locations, each derivative set of the first constituent array arranged as a row of locations, and each derivative set of the second constituent array arranged as a column of locations.

An embodiment of the invention may also include, for at least one constituent array, each location of any derivative set containing at least one entity, all locations of a particular. derivative set in the at least one constituent array containing substantially the same concentration of constituent composition. The embodiment may further include that each entity in a given derivative set of one constituent array be present in another derivative set of every other constituent array. The embodiment may also further include a combination of entities that is only present in one derivative set for all constituent arrays. Optionally, the embodiment may also include that each entity in the combination not be present with any other entity of the combination in any other location of any other constituent array.

Another method for evaluating the activity of a set of combined compositions, consistent with an embodiment of the invention, includes the step of providing, for each constituent array, a composition control in each location of a composition control set of such array, wherein the composition control set of each constituent array is disposed so that all locations of the composition control set of a given constituent array are distinct from any locations of such constituent array that correspond to locations of the composition control set in any other constituent array. At least one of the composition controls may be a positive control, and at least one of the composition controls may be a negative control. The method may also include the steps of performing statistical analysis on the measured values of activity in a location holding a constituent control to provide a measure of data quality associated with an array. A particular method may include the steps of providing a standard deviation value and an average value, either numerical average or median value, for each set of positive control locations and negative control locations of a composition control set for each physically distinct object of an assay array, the values based upon the activity in locations of the composition control set; and providing a z-factor for each physically distinct object of the assay array based upon the standard deviation values and the average values. Alternatively, the method may include the steps of providing a local quantized c-value, determined for particular locations of a composition control set of a physically distinct object of an assay array, a local quantized c-value being dependent upon a fractional value of activity for the particular location, the fractional value of activity being a value of the activity at the particular location relative to a normalization value; and providing a global c-value for each physically distinct object of the assay array based upon a numerical average of the local quantized c-values for the particular locations of the physically distinct object of the composition control set. The normalization value may be a measured activity level in a location with an expected activity level of zero, a measured activity level in a location with no test entity, or a selected activity value.

An alternate method of an embodiment of the invention, wherein each location of any constituent array has a corresponding location in any of the other constituent arrays, further includes providing an assay control in each location of an assay control set of an assay array such that the location of the assay control set in the assay array has a corresponding location in each constituent array. The locations of the assay controls may be distributed anywhere on an assay array, and may include a location adjacent to the edge of a plate, when plates are utilized as an array. The locations may also be arranged from one end of a physical entity holding a portion of the assay array to another end. The assay controls may be provided in one or more corresponding locations of a constituent array before providing the assay array.

In a related embodiment of the invention, a method for evaluating the activity of a set of combined compositions includes evaluating a measured activity of the assay control in each location of the assay control set; providing a deviation activity value for a plurality of locations of the assay array based upon the measured activity and an expected activity in one or more locations of the assay control set; and assigning a corrected activity value for each of the plurality of locations of the assay array based upon the deviation activity values. The plurality of locations of the assay array may have the same expected value of activity. As well, providing the deviation value may include providing interpolated values based upon the measured activity in one or more locations of the assay control set.

In another related embodiment of the invention, a method of evaluating the activity of the combined composition includes identifying erroneous activity values in one or more locations of the assay array; and assigning a replacement value of activity in each location associated with the erroneous activity value. The replacement values may be assigned based upon the evaluated activity in one or more adjacent locations relative to the location associated with the erroneous activity value, or the concentration of at least one constituent composition in one or more adjacent locations relative to the location associated with the erroneous activity value.

Further alternate embodiments of the invention may include providing a dilution array of locations, each location of the dilution array corresponding to a particular member of the set and being associated with a designated aliquot from each of the constituent arrays, wherein each aliquot is one of zero and non-zero, and deriving the assay array of locations from the dilution array. A concentration of a particular entity in a location of the dilution array may be at least approximately one order of magnitude more dilute than the concentration of the particular entity in a designated constituent array. As well, a concentration of a particular entity in a location of the assay array may be at least approximately one order of magnitude more dilute than the concentration of the particular entity in a designated dilution array.

Another alternate embodiment of the invention includes providing the origin set and corresponding derivative sets of a constituent array on distinct physical objects. The embodiment may further provide for the assay array to be embodied in a plurality of distinct physical objects.

Other embodiments of the invention are directed toward facilitating the evaluation of activities of combined compositions. In one embodiment, the evaluated activity of each location of an assay array is expressed in terms of inhibition. The inhibition may also account for the background signal associated with a particular type of measurement. Background signals may be based upon a measured activity in a location with an expected activity level of zero, a measured activity in a location with no test entity, or as assumed value of zero. Background signal may be based upon measurement in one location, or an average of a plurality of locations; the locations may contain a control. Locations for measurements of an untreated value, utilized in calculating inhibition, may also be based upon one or more locations.

In another embodiment of the invention, a method for evaluating the activity of a set of combined compositions includes providing a measure of synergy for a plurality of members of the set, the measure of synergy depending upon a measured value and a predicted value for each location of the set, each measured value being pertinent to the activity in one location of the set, and each predicted value being calculated from a model. The model may depend upon measured values pertinent to an activity of at least one entity of a candidate composition in the one location of the set. As well, the predicted values may be the activity of the at least one entity of the candidate composition. Alternatively, the predicted value may be calculated from the Bliss Independence Model or the Loewe Additivity Model. The measure of synergy may be a difference between a measured value and a predicted value for each location of the set. Another measure of synergy may be the sum of the difference between the measured value and predicted value for a plurality of locations of the set. Yet another measure of synergy may be a representation of the concentrations of entities in a candidate composition associated with a specific level of activity derived from interpolation of a plurality of measured values. Evaluating the activity may also include replacing particular measured values with calculated values that maintain a smooth monotonically changing surface of values with respect to each calculated value and measured values at locations adjacent to the calculated value.

Another embodiment of the invention involves a method of evaluating the activity of a set of compositions in an array. The method comprises determining a measured value for each location of a set of compositions, for each of a plurality of sets of the array, pertinent to the activity thereof, wherein each set of the array includes substantially the same set of compositions arranged in corresponding locations; for each of the locations of the sets of the array, determining predicted values of activity according to each of a plurality of models; and determining the activity of the set of compositions based upon the measured values and predicted values using at least one statistical method. Determining the activity may include determining the activity based upon the difference between the measured value and the predicted value in corresponding locations of each set for each of the plurality of models, or providing a summation of all difference values exceeding a difference threshold for each set of the array. The use of one statistical method may include determining a standard error of activity associated with a location of a set based upon the measured values in corresponding locations of each of the plurality of sets of the array. Such standard errors may be used to determine a measure of error of the activity of the set (e.g., using the standard errors to determine a square-root of the sum of the squares of the standard errors of activity of the plurality of locations). Use of a statistical method may also include determining an average measured value associated with a location of a set based upon the measured values in corresponding locations of each of the plurality of sets of the array, or determining a ratio of an average measured value to a standard error associated with a location of a set based upon the measured values in corresponding locations of each of the plurality of sets of the array.

In an alternate embodiment of the invention, values of the evaluated activity in an assay array are extrapolated or interpolated to provide predicted values of the evaluated activity at combined concentrations that are not measured directly from the assay array. The embodiment may be utilized to predict the set of candidate composition values that are expected to result in a chosen activity level. The embodiment may also be used to identify erroneous measured values of evaluated activity in an assay array; the interpolated or extrapolated values may be used in place of the measured erroneous values.

Other embodiments of the invention are directed toward assay arrays and constituent arrays that are utilized in the methods herein described. Some embodiments of the invention are also directed toward computer program products for evaluating a combination effect following the methods described herein.

### BRIEF DESCRIPTION OF THE DRAWINGS

The foregoing features of the invention will be more readily understood by reference to the following detailed description, taken with reference to the accompanying drawings, in which:

FIG. 1 illustrates diagrammatically an embodiment of the invention that uses constituent arrays that hold constituent compositions and their combination to form an assay array holding combined compositions;

FIG. 2 illustrates diagrammatically an embodiment where each array location has at least one corresponding location in every other array;

FIG. 3 illustrates diagrammatically an embodiment of the invention related to the making of an assay array utilizing an intermediate dilution array;

FIG. 4 illustrates diagrammatically embodiments of the invention related to possible configurations of constituent arrays, including the use of origin sets and derivative sets in a given constituent array;

FIG. 5 illustrates diagrammatically an embodiment of the invention that shows a configuration of a particular constituent array in which the origin set is provided on a different physical object from the derivative set;

FIG. 6 illustrates diagrammatically an embodiment of the invention related to a method for testing the activity of a plurality of entities simultaneously in an expedited fashion;

FIG. 7 illustrates diagrammatically an embodiment of the invention related to the possible configurations of constituent arrays that include locations for composition controls and assay controls;

FIG. 8 presents some examples of embodiments of the invention utilizing possible configurations of constituent arrays that include blocks of locations holding combined compositions, and locations for composition controls and assay controls;

FIG. 9 illustrates diagrammatically stages of the data process of recalculating data from an assay array to account for plate effects, in accord with an embodiment of the invention;

FIG. 10 illustrates an embodiment of the invention in the diagram of a 6×6 assay having data related to the evaluated activity of the combined compositions presented in three forms: inhibition, the difference between the inhibition and the highest single agent, and the difference between the inhibition and the Bliss Independence Model;

FIG. 11, in accord with an embodiment of the invention, illustrates two depictions of a data set having 6 blocks of 6×6 locations: (A) before spike filtering; (B) after spike filtering;

FIG. 12 presents, in accord with embodiments of the invention, a diagrammatic representation of a comparison between the inhibition vs. concentration curves for a set of combined compositions, a Bliss Independence Model, the single agents of the combined composition, an average curve for the set of combined compositions, and the spread in set of data of combined compositions and the difference between the average curve and the Bliss Independence Model;

FIG. 13 illustrates two graphs of the evaluated activity of an assay array presented in terms of inhibition and the ratio of the difference of average inhibition and the highest single agent to the deviation of the of the set of inhibition determinations, in accord with embodiments of the invention;

FIG. 14 provides illustrations showing the results of assaying various mixtures of chlorpromazine and cyclosporine A, utilizing embodiments of the invention, for the suppression of phorbol 12-myristate 13 acetate/Ionomycin stimulated IL-2 and TNF-α secretion from human white blood cells using the ELISA method, the illustrations depicting the single agent inhibition as a function of concentration; the mean inhibition at locations of the assay array; the standard error associated with locations of the assay array; the difference between the measured inhibition and the predicted inhibition from a highest single agent model for locations of the assay array; the difference between the measured inhibition and the predicted inhibition from a highest single agent model for locations of the assay array; and an isobologram of the 80% inhibition for various concentrations of the mixtures using the measured results and the results expected from the Loewe Additivity Model.

FIG. 15 illustrates an X constituent array of compositions utilized in Example 2, in accord with embodiments of the invention;

FIG. 16 illustrates a Y constituent array of compositions utilized in Example 2, in accord with embodiments of the invention;

FIG. 17 illustrates an assay array derived from the combination of the X and Y constituent arrays of Example 2, in accord with embodiments of the invention;

FIG. 18A illustrates an assay array of combined compositions A and B over a range of concentrations of A and B, in accord with an embodiment of the invention;

FIG. 18B illustrates an assay array of combined compositions A and B, wherein the range of concentrations of A and B are selected based upon the transition zone activity of composition A and composition B, in accord with an embodiment of the invention;

FIG. 19 illustrates two arrays configured to create a combination array with locations corresponding to a virtual sparse assay array, in accord with embodiments of the invention;

FIG. 20 illustrates an assay array, in accord with embodiments of the invention, resulting from the combination of the constituent arrays of FIG. 19, and representations of virtual sparse assay arrays of two combined constituent compositions of the assay array;

FIG. 21 illustrates the results of a simulation of automated synergy identification of existing data concerning 92 pairs of constituent compositions at a variety of concentrations, the graph being a plot of the percentage of manual hits corresponding to synergetic combination found by the automated method as a function of the top n % of combinations examined of the assay array, the assay arrays being (i) an assay array of data in which every concentration of a constituent composition was combined with every concentration of every other constituent composition in the assay array; (ii) the assay array of (i) in which locations of data are only examined that correspond to a sparse array configuration of (i). A plot of the probability of random guessing is also included.

FIG. 22 illustrates the results of an automated synergy identification of a pilot experiment involving 92 pairs of constituent compositions at a variety of concentrations that resulted in the manual identification of 22 synergistic combinations. The graph illustrates the number of the synergistic combinations that were identified as a function of the top n % of scored combinations searched according to two screening methods. One method provides an assay array in which every concentration of a constituent composition was combined with every concentration of every other constituent composition in the assay array. The second method provides an assay array with locations corresponding to a virtual sparse array that combines every concentration of every other constituent composition in the assay array. The second method also employs concentration selection based upon the activity of the pure constituent compositions. A plot of the probability of random guessing is also included.

FIG. 23 illustrates an assay array, in accord with embodiments of the invention, including six 6×6 arrays in which concentration selection and correspondence to a virtual sparse assay array is not utilized;

FIG. 24 illustrates an assay array, in accord with embodiments of the invention, that combines a constituent array configured to create an assay array corresponding to a virtual sparse assay array and a constituent array configured as a column array having a plurality of entities at a high concentration;

FIG. 25 illustrates two constituent arrays, in accord with embodiments of the invention, configured to create an assay array, the constituent arrays configured to contain pair of rows or columns having a constituent composition;

FIG. 26 illustrates the assay array resulting from combining the two constituent arrays of FIG. 25, and representations of virtual sparse assay arrays of combined constituent compositions B and F of the assay array, in accord with embodiments of the invention; and

FIG. 27 illustrates a three dimensional virtual sparse assay array configuration, in accord with embodiments of the invention.

### DETAILED DESCRIPTION OF SPECIFIC EMBODIMENTS

Definitions. As used in this description and the accompanying claims, the following terms shall have the meanings indicated, unless the context otherwise requires:

An “activity” of a composition is a change in state of at least one entity of the composition. The activity is usually determined relative to a change in state of a test entity, wherein the test entity's change in state is due to the presence of a candidate composition.

An “aliquot” is an allotment of one or more compositions from a particular set of compositions.

An “array” is an object capable of holding one or more compositions, wherein each composition is held separately from any other composition for evaluation. Each array has a set of locations corresponding to the position where a discrete composition may be located. An array may be embodied as a plate, the plate having a plurality of wells or microwells; plates having 96 wells, 384 wells, 1536 wells, or other high density assay plates may be utilized, though every well of a plate is not necessary utilized in the array. An array may also be embodied as a flat impermeable substrate with a number of locations where small amounts of composition are deposited. An array may also be embodied as a substrate that is porous or penetrable, having locations that are associated with a particular sample (as described, for example, in U.S. Patent Application 2003/0032203 A1 of Sabatini et al.); or a microvolume conduit (as described, for example, in U.S. Patent Application 2002/0151040 A1 of O'Keefe et al.). An array may also be embodied as more than one physically distinct object. FIG. 2 provides an illustration of an array 210 that is embodied as three separate physical objects. In many of the embodiments of the invention described herein, the arrays are embodied as plates with a well at each location, though practice of the embodiment is not limited to the use of plates with wells.

An “assay” array is an array (as defined above) holding a set of combined compositions.

An “assay” control is a control (as defined below) utilized in an assay array.

A “candidate” composition is a composition (as defined below), including a subset of a composition, essentially consisting of one or more entities that affect the activity of a combined composition.

A “candidate” entity is an entity (as defined below) that affects the activity of a combined composition.

A “composition” is a set of one or more entities that constitute a discrete sample. Each composition may include the same or a different set of entities, compared with any other composition. The absolute amount and concentration of a particular entity within a composition may match or differ from the absolute amount or concentration of the entity in any other composition. Thus two compositions can be the same, though they differ in the concentration or quantity of one or more entities.

A “combined” composition is a composition (as defined above) formed from combining a plurality of members of constituent compositions.

A “concentration” of a particular constituent composition refers to the concentration of one entity or a combination of a plurality of entities in a particular constituent composition.

A “constituent array” is an array (as defined above) holding a set of constituent compositions.

A “constituent” composition is a composition (as defined above) utilized to make a combined composition.

A “composition” control is a control (as defined below) utilized in a constituent array, which may be transferred to an assay array. The composition control may be a substance associated with a particular entity of a constituent array. The composition control may be utilized to detect errors in an array, and to help insure quality control of any data evaluated in an assay array.

A “control” is a substance with a known, expected activity.

A “derivative” set of locations is a set of locations in an array corresponding with one particular location of an origin set, wherein each derivative set location contains an aliquot from the particular origin set location.

A “diluent” is one or more entities of a composition that does not substantially affect an activity of a composition other than through the diluent's effect on the concentration of a composition.

An “entity” is a component of a composition. Types of entities utilized in a combined composition include components of an evaluative composition, such as a test entity; components which act to change the state of a test entity in a composition, herein known as “candidate” entities; and components which do not affect the activity of an evaluative composition other than through how their presence affects the concentration of the composition, herein known as diluents. Some non-limiting examples of specific entities include a chemical substance; a drug; a biological moiety; and a substrate capable of holding a chemical substance, drug, or biological moiety (e.g. small polymeric particles with an absorbed layer of an organic molecule). An entity may be a component of an assay for analysis of a compound, or may be the compound itself or a component of the compound.

An “evaluative” composition is a composition (as defined above) that aids or enables evaluation of the activity of a composition.

A “negative” control is a control (as defined above) with an expected activity that is typically zero. For example, a substance with a known and expected ability not to suppress cell production of a metabolic product may serve as a negative control wherein activity is measured as the ability to suppress the production of the metabolic product.

An “origin” set of locations is a set of locations in an array wherein each location is associated with a unique derivative set of locations in the array.

A “positive” control is a control (as defined above) with an expected activity that is typically greater than zero. For example, a substance with a known and expected ability to suppress cell production of a metabolic product may serve as a positive control wherein activity is measured as the ability to suppress the production of the metabolic product.

A “set” is a group with at least one member.

A “test” entity is an entity (as defined above) which undergoes a change of state when exposed to a particular candidate entity or candidate composition.

Embodiments of the invention provide methods for evaluating the activity of a set of combined compositions created by combining a plurality of constituent compositions. Specific embodiments create and organize constituent and combined compositions. These embodiments may facilitate accelerated evaluation of the activity of the combined compositions, or improve the accuracy of determining the activity of the combined compositions, while evaluating the activity of the set in a reliable, data-rich manner. For example, some embodiments of the invention may allow the evaluation of more than half a million combinations of entities with varying components and concentrations using several assay arrays.

Embodiments of the invention described herein are intended to be merely exemplary and a number of variations and modifications will be apparent to those skilled in the art. All such variations and modifications are intended to be within the scope of the present invention. Though embodiments of the invention described herein have particular relevance to the field of drug evaluation and discovery, some embodiments of the invention will find application in other fields that utilize combinatorial testing or the evaluation of a large number of samples. A few non-limiting examples of such fields include catalyst discovery and evaluation; methods of chemical synthesis and analysis; and evaluation of the benefits or toxicity of a mixture or chemical upon a given biological moiety.

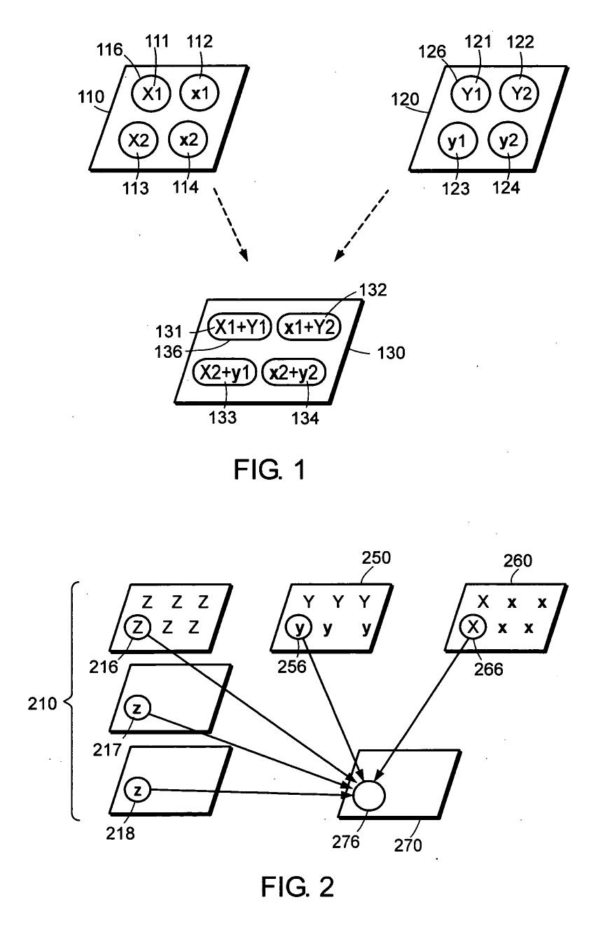

Some features of embodiments of the invention will be more readily understood by reference to FIG. 1, which shows constituent compositions 111, 112, 113, 114, 121, 122, 123, 124, held by constituent arrays 110, 120 being combined to form combined compositions 131, 132, 133, 134 held by an assay array 130. The activity of each combined composition 131, 132, 133, 134 is evaluated. In FIGS. 1 and 2, each alphanumeric code, for example X1 or Z, refers to a specific constituent composition, regardless of whether the letter is uppercase or lowercase; codes with an uppercase letter represent candidate compositions of a higher concentration of a candidate entity than a similar code using a lowercase letter. For example, Y1 has the same constituent composition as y1, though y1 has a lower concentration of at least one of the entities of the constituent composition.

In the context of drug discovery, a novel drug may be created from a combination one or more known drugs (sometimes called herein a “candidate composition”) with other compounds, wherein the drugs acting together produce an effect differing from the expected effects of the individual drugs taken in isolation (sometimes called herein a “combination effect”). Some embodiments of the invention may help identify such combination effects. When the combination has an effect greater than the combined expected effect of each drug acting independently, the combination has a synergistic effect. When the combination has an effect less than the combined expected effect of each drug acting independently, the combination has an antagonistic effect. Other possible examples of novel drug combinations include identifying one or more drugs that counteract the side effect that a particular drug typically exerts on a test entity; or identifying one or more drugs that counter a negative effect that a particular drug exerts on a test entity (e.g. toxicity due to the particular drug).

Combination effects of a candidate composition may also be due to the formation of interaction networks involving complex connections between many components, wherein the components are typically known to interact with specific molecular targets but the combination exhibits a pleiotropic effect. Thus, embodiments of the invention may also identify unknown interactions in an interaction network by identifying the synergism or antagonism present in a mixture; provide information of the connectivity of disparate interaction networks by helping identifying correlations between a candidate composition's synergism and the relationship of the composition's components; and help determine the dependence of the proximity in the pathway of the components' known targets on the strength of the degree synergy or antagonism in a candidate composition when the pathway is well understood.

Any candidate composition may include a substance approved by a governmental entity, such as the U.S. Food and Drug Administration, for administration to a patient. Alternatively, the candidate composition may include at least two entities, each approved of by a government entity for administration to a patient. The candidate entities may also be drugs approved of by a governmental agency and having at least one of an established safety profile, a recognized pharmacology profile, and a recognized toxicity profile. Moreover, the candidate composition may also be a combination wherein each component drug has little to no effect when taken individually, but the component drugs produce a substantial effect when the components are taken in tandem. Of course, candidate compositions utilizing a substance approved for use by a government entity for administration to a patient may include other entities which have not received such governmental approval.

Though candidate compositions may oftentimes involve two or fewer candidate entities in a combined composition, candidate compositions may also include three or more candidate entities in embodiments of the present invention. Likewise, embodiments of the invention may include a candidate composition with only one candidate entity.

Ultimately, systems and methods in accordance with embodiments of the present invention are concerned with evaluating the activity of a candidate composition, i.e. evaluating the affect a candidate composition has upon some the state of a particular entity. To evaluate the activity, typically a candidate composition is exposed to an evaluative composition having one or more test entities; the combination of evaluative composition and candidate composition comprise a combined composition. Thus, one way of evaluating the activity of a combined composition involves measuring the change in some state of an entity in the combined composition, such as a test entity, that is exposed to a candidate composition. Combined compositions, as well as constituent compositions, may also include diluents as one or more additional entities to control the concentration of a particular entity in a composition.

Examples of entities utilized in evaluative compositions include components of a disease-model assay, cytoblot assay, a reporter gene assay, components of a florescence resonance energy transfer assay, a fluorescent calcium binding indicator dye, or components used in either fluorescence microscopy or expression profiling. These techniques are detailed more thoroughly in PCT application “Methods for Identifying Combinations of Entities as Therapeutics,” International Publication Number WO 02/04949 A2, the relevant portions of which are hereby incorporated by reference. Test entities within an evaluative composition may include one or more types of cells, tissues, animals, reconstituted cell-free media, and one or more biologically relevant molecules such as a protein or an oligonucleotide. A test entity in a composition may also act as a component of an evaluative composition while simultaneously inducing a change in activity in another entity of a composition, i.e. also being part of the candidate composition.

The change in state of a particular entity, or test entity, typically refers to some effect that a candidate composition may have on the particular entity; this state may also be affected by other environmental factors, for example temperature, pressure, or light/radiation exposure. The effect may be through individual interactions of the entities of a candidate composition with the entity, or through an interaction of the entity with the entire combination of the candidate composition. The specific measure of change of state depends upon what characteristic in the particular entity may be altered by the presence of a candidate composition. In the specific instance where the change of state is identified for a test entity, such as a particular type of cell, the change in state may refer to cell interactions or metabolism. Non-limiting examples include measuring the products of DNA synthesis; measuring the production of a particular metabolic product of a cell type; measuring the overall effect on anti-proliferative activity, or cell viability, of one or more types of cells; or measuring a change in one or more aspects of cell morphology.

Changes in state of a particular entity by a candidate composition may be influenced by one or more interactions between entities within a candidate composition, as well as the interaction between the candidate composition (acting as individual components or collectively) and the particular entity. Non-limiting examples of the interactions include the effects derived from separate individual effects of each of the constituent entities on a test entity (e.g. independent non-networked effects of two or more compounds on a cell); the combined effect of a candidate composition on a test entity (e.g. each entity of a candidate composition acts upon different portions of an interaction network or pathway); or by the interaction between constituent entities of a candidate composition to create another new entity that effects a test entity (e.g. a chemical reaction, or a physical association, between entities in a candidate composition to create a new entity, where the location of an assay array acts as a vessel for the transformation). The particular mechanisms by which a change in state is achieved, however, do not affect the practice of embodiments of the invention, however, since the embodiments are directed to evaluating the activity of combined compositions regardless of how the change in state of an entity is achieved.

Creating Combined Compositions and Assay Arrays

Referring to FIG. 1, an assay array 130 holds a set of combined compositions 131, 132, 133, 134 derived from a plurality of constituent arrays 110, 120. Each combined composition 131 is positioned in a particular location of an assay array 136. The combined composition 131 is formed by combining a member from each of a common plurality of constituent compositions 111, 121. Each set of constituent compositions is physically associated with a constituent array 110, 120, each constituent composition 111, 121 located in a particular location 116, 126 of its associated constituent array.

Particular constituent compositions, utilized to form a combined composition, may be composed solely of an evaluative composition, a candidate composition, or one or more diluents. Alternatively, a constituent composition may consist of any combination of compositions and diluents.

Constituent arrays may be embodied as a plate with wells, each well containing a constituent composition of the constituent array. Constituent arrays may also be embodied as a single source container with a single composition. For example, a constituent composition and constituent array may be embodied as a diluent from a container; the diluent is subsequently added into the wells of an assay array plate holding a combined composition. One constituent array may also be embodied as multiple sources, each containing one or more entities of a composition. For example, a constituent composition may be an evaluative composition which is inserted into each well of an assay array plate, the constituent array embodied as sets of entities of the evaluative composition contained in a plurality of source containers.

The combining of constituent compositions in constituent arrays to form a combined composition in an assay array may be performed in any manner known in the art. For example, with respect to embodiments of the invention utilizing plates with wells, constituent compositions in wells of plates of constituent arrays may be pipetted manually from corresponding wells in constituent array plates to a well of an assay array plate. In high throughput screening applications, the combining of constituent compositions in wells of a plate may be facilitated by the use of automated machinery such as the Packard Mini-Trak (PerkinElmer Life Sciences Inc., Boston Mass.). Automated machinery may combine compositions from constituent arrays on a well-by-well basis, or by combining a plurality of wells substantially simultaneously in order to decrease processing time.

In a particular embodiment of the invention, each location of each array is associated with at least one corresponding location in every other array. Referring to FIG. 1A, an embodiment of the invention is shown where each array 110, 120, 130 is embodied as a single plate with wells arranged in a 4×4 square matrix. Aliquots from each constituent composition 111, 112, 113, 114, 121, 122, 123, 124 of each constituent array 110, 120 are combined in a geometrically corresponding location of the assay array 130 to form a set of combined compositions 131, 132, 133, 134. In FIG. 2, assay array 270 is formed from combining constituent arrays 210, 250, 260. In particular, location 276 of the assay array has corresponding locations 216, 217, 218, 256, 266 in each of the constituent arrays 210, 250, 260. Likewise, locations 216, 217, 218 of constituent array 210 have corresponding locations 256, 266 in constituent arrays 250, 260 and assay array 270. Aliquots of compositions in each of the corresponding locations of the constituent arrays 216, 217, 218, 256, 266 are combined in a location of the assay array 276 to form the corresponding combined composition.

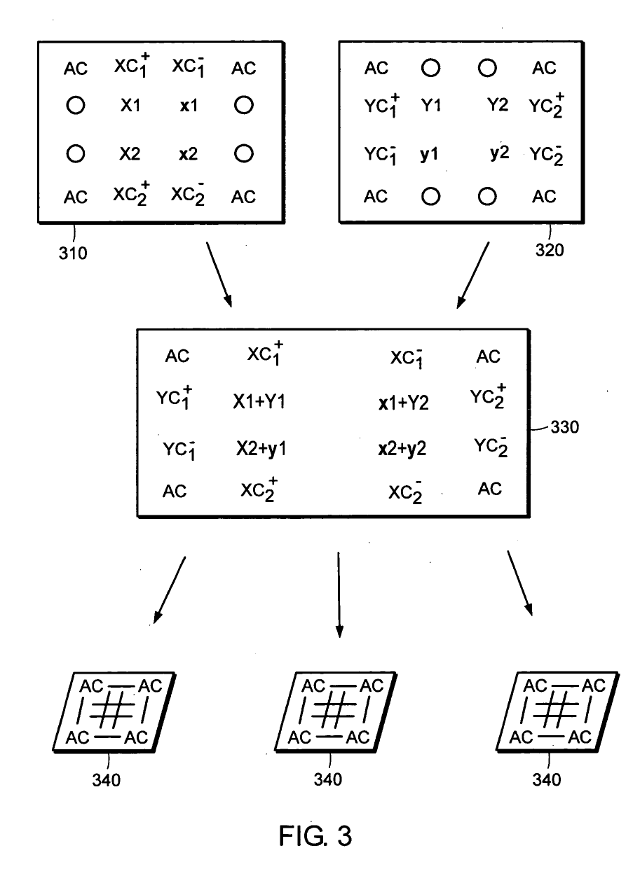

An assay array may be embodied as more than one physically distinct object. For example, an assay array may comprise several plates of combined compositions wherein each plate is substantially identical, i.e. having the same combined compositions in the same concentration and quantity, the combined compositions arranged similarly on each plate. Referring to FIG. 3, in an embodiment of the invention, constituent compositions on constituent arrays 310, 320 may be combined in any means described herein or known in the art, to form combined compositions on a dilution array 330. The embodiment may be practiced with the condition that a specific entity in a location of the dilution array is at least approximately one order of magnitude more dilute than the concentration of the specific entity in a designated constituent array. Each location of the dilution array 330 has at least one corresponding location in an assay array 340. As depicted in FIG. 3, aliquots from each location of the dilution array 330 are deposited into corresponding locations of the assay array 340 to form the combined compositions in the assay array 340. In a particular embodiment of the invention, a plurality of locations of the assay array contains at least one entity from the corresponding location of the dilution array in which the entity's concentration in the assay array is substantially one order of magnitude more dilute than the concentration in the dilution array. The dilution in the assay array may be facilitated by the use of a diluent in each location of the assay array. Utilization of a dilution array may facilitate the production of a large number of plates for evaluating a composition, corresponding to an assay array, without repeated combining of constituent arrays.

In the aforementioned embodiment, each of the physically distinct objects of an assay array need not be substantially identical in compositions or arrangement of compositions. For example, different plates of an assay array may contain differing types of evaluative compositions added to each well of a particular plate in order to test varying types of activity associated with the combined compositions. In another example, the combined compositions in different plates may have differing dilutions, though the plates contain the same composition.

Creating Constituent Compositions and Constituent Arrays

Constituent arrays may be created in any manner known in the art. Manual pipetting of entities into each location of a constituent array from various source containers provides one possible example. For applications requiring higher throughput, automated machinery may be employed to increase speed and accuracy of array creation. Machines such as the Packard Multi-Probe (PerkinElmer Life Sciences Inc., Boston, Mass.) may be used to enable automated transfer of entities in source vials to wells of a constituent array plate.

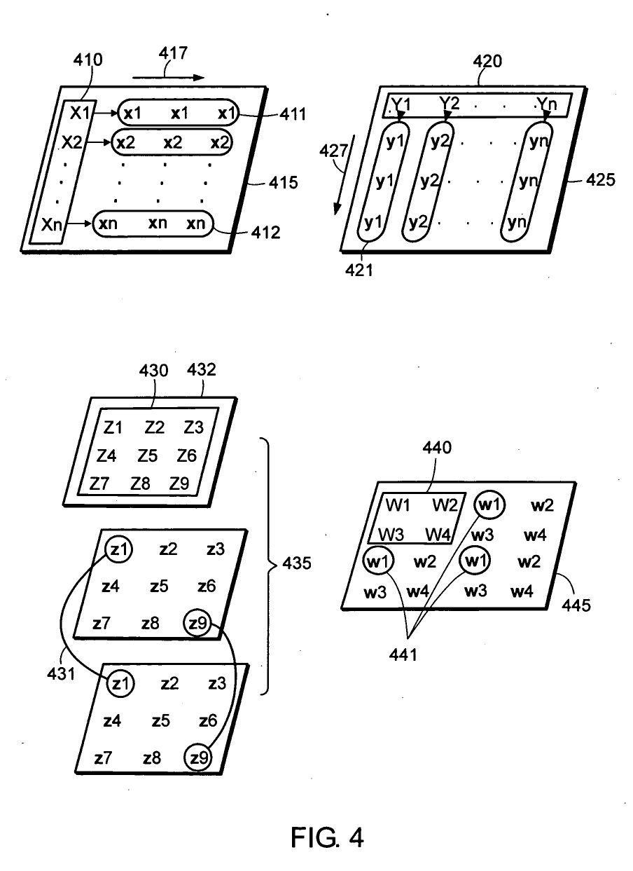

Evaluating the activity of a large number of combined compositions may be facilitated by arranging the locations of compositions on the constituent arrays or assay array in particular configurations. The configurations may increase the speed of producing arrays, while insuring the quality of data related to evaluating the activity of combined compositions. FIG. 4 illustrates diagrammatically several embodiments of configurations that may be utilized for constituent arrays.

In an embodiment of the invention, some examples of which are depicted in FIG. 4, a set of locations in a particular constituent array form an origin set 410, 420, 430, 440. The origin set may be embodied on the same physical object as the remainder of the constituent array as depicted by arrays 415, 425, 445, or may be embodied on a separate object relative to the rest of the constituent array as depicted by array 435. Each member of the origin set has a corresponding set of one or more unique locations of the constituent array, which are known as a derivative set 411, 412, 421, 431, 441. As shown in FIG. 4, each origin set location and its corresponding set of derivative locations are designated with the same alphanumeric label, origin locations marked by capital letters and derivative locations marked by lowercase letters. For example, in constituent array 425 the location marked Y1 represents an origin location, while locations marked by y1 represent derivative locations corresponding with the origin location Y1; thus the set of locations 421 is the derivative set associated with Y1. Analogously, for the constituent array 435 embodied as three separate plates, the set of locations 431, each location designated by z1, is the derivative set corresponding with origin set location Z1 on 432.

The members of a particular derivative set may also be embodied on one or more physical objects. Each location of a derivative set contains a composition with the same set of entities as the composition in the associated location of the origin set. In a particular embodiment, the composition in each derivative set location may be derived directly from the associated origin set location, e.g. an aliquot from the origin set location. Furthermore, the set of locations constituting an origin set may be embodied on a single physical entity.

The constituent arrays depicted in FIG. 4 combine all the features discussed in the above paragraph. In arrays 415, 425, 445, the origin set and associated derivative sets are all embodied on one plate, while the array depicted by 435 utilizes the origin set on a single plate with the corresponding derivative sets having one member on each separate physical entity.

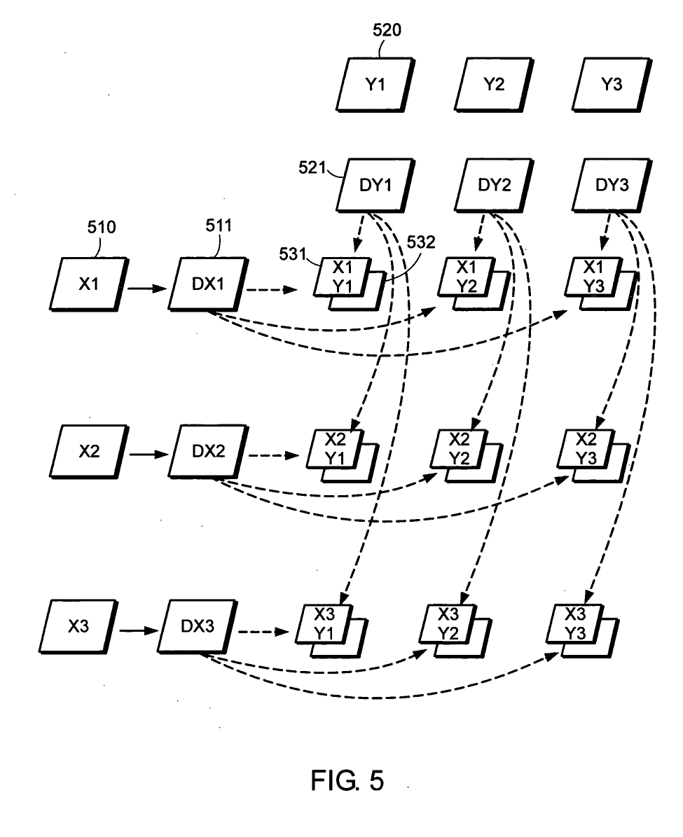

The constituent array configuration depicted array 435 may further be used to create a series of intermediate objects that are subsequently combined to create an assay array. In a separate embodiment of the invention, compositions held by derivative sets of constituent arrays are combined to form combined compositions corresponding to an assay array. This embodiment may allow the repeated use of origin sets, each embodied on a separate physical object, to enable the creation of a large number of different combined compositions on assay arrays. An example of such an embodiment is depicted in FIG. 5. Origin sets 510, 520, drawn to separate constituent arrays, are each embodied on a separate physical object. The origin sets 510, 520 may be created in any manner, including utilizing the steps of making a particular embodiment of a constituent array 415, 425, 445 as depicted in FIG. 3. Derivative sets 511, 521 are defined in the embodiment such that each location of a derivative set corresponds with one location of the corresponding origin set 510, 520, respectively. Each derivative set 511, 521 holds a composition including an aliquot from the corresponding location in the origin set 510, 520. The compositions from the derivative sets 511, 521 may be combined to form an assay array, which is embodied as several separate objects 531, 532 that are each formed from combining derivative sets 511, 521.

The aforementioned embodiment may provide the additional advantage of protecting constituent arrays from possible cross contamination since the derivative sets 511, 521 are utilized in creating multiple assay arrays with different combined compositions as shown in FIG. 5. The origin sets 510, 520 are less subject to contamination since they are only utilized to make an array with the same composition. Also, contamination of the derivative sets may be rectified by creating new derivative sets from the origin sets.

In another embodiment of the invention, a constituent array is created which provides for compositions in which one or more entities are serially diluted. Use of this embodiment facilitates the testing of a range of concentrations of a given entity to evaluate, for example, the change in state of a test entity relative to the concentration change of a candidate entity in a composition. The embodiment requires successive dilutions of an entity for each location of a given derivative set. In an example, referring to FIG. 4, derivative group 411 contains a set of locations in which a particular composition, X1, becomes more dilute in each location as the wells are located further down the row in the direction 417. Similarly, the locations of derivative group 421 contain a more dilute concentration of a composition, Y1, as wells are located further down the column in direction 427.

Each individual derivative set may carry serial dilutions of a particular entity; each set may or may not serially dilute the same entity as any other set. In a particular embodiment, aliquots from an origin set location are deposited to corresponding locations of the derivative set; the aliquots may be either the same of differing quantities for each location of the derivative set. The successive dilutions in each location of a derivative set may be achieved adding a diluent, or other entities, in varying quantities to a plurality of members of the derivative set. The precise quantities of composition from the origin set, diluent, and other entities to be added to each location of a derivative set depend upon the range of concentration and change in concentration per location desired by a user.

In an alternate embodiment utilizing serial dilutions in successive locations of a derivative set, the dilution of an entity of a composition may proceed in steps of approximately a fixed multiple relative to another location in the derivative set. In a first particular alternate embodiment, the members of the derivative set may span a concentration range of a factor of at least approximately 50,000, achieved in steps of a factor of at least approximately three between derivative set locations. In a second particular alternate embodiment, the members of the derivative set may span a concentration range of a factor of at least approximately 1,000, achieved in steps of a factor of at least approximately four between derivative set locations. In a third particular alternate embodiment, the members of the derivative set may span a concentration range of a factor of at least approximately 250, achieved in steps of a factor of at least approximately two between derivative set locations. Though these embodiments describe a particular range of concentration and step change of concentration per location, one skilled in the art would recognize that serial dilutions of a derivative set may be carried out over any number of ranges of concentration using a variety of step changes of concentration per location of interest.

Creation of constituent arrays utilizing origin and derivative sets may be performed using any technique known in the art. One technique that may be utilized is manual pipetting of compositions into the origin set locations, followed by creating serial dilutions in the associated derivative set locations derived in part from aliquots of the corresponding origin set location. Automated machinery utilizing the concepts of origin and derivative sets may expedite the creation of constituent arrays. Machines such as the Packard Multi-Probe may be used to transfer entities to origin set locations in order to create compositions in the locations. Serial dilution of the compositions as added to locations of corresponding derivative sets may be performed using machinery such as the Tomtec Quadra Plus (Tomtec Inc., Hamden, Conn.).

The aforementioned embodiments of the invention utilizing origin sets and derivative sets may be particularly advantageous in aiding the identification of combined compositions that have an activity that depends particularly on the relative concentration of particular entities in the combined composition. Consider a situation where each array is embodied as one plate having a fixed number of wells configured in evenly spaced rows and columns, with the geometrically similarly located wells of each array corresponding to each other. Referring again to FIG. 4, consider a situation in which a constituent array 415 is created with a set of compositions in origin locations 410, each composition being serially diluted with respect to a candidate entity in corresponding derivative locations 411, 412 with adjacent locations of a derivative set becoming more dilute in the candidate entity as locations proceed in direction 417. Let the set of constituent compositions be denoted as C. If a second constituent array is created with a configuration similar to array 415, with the set of constituent compositions of the second array being denoted as D, the trends of serial dilution for candidate entities in compositions C and D will follow one another when a combined composition is formed from constituent compositions C and D. Evaluating the activity of the combined compositions created from such a configuration of constituent arrays increases the difficulty of determining whether a change in activity is affected more by the presence of a candidate entity associated with composition C or composition D; this is because the concentration gradient of candidate entities in wells for compositions C and D will correspond in the assay array wells. An advantage may be obtained by creating combined compositions formed from a particular concentration of a candidate entity in composition C with a range of concentrations of a candidate entity in composition D, and visa versa.

Thus, in another embodiment of the invention, given that each location of each array has at least one corresponding location in every other array, the constituent arrays are configured such that more than one location from an origin set location and its corresponding derivative set locations in a given constituent array, is distinct from the corresponding locations of a combination of an origin set location and its corresponding derivative set locations in any other constituent array. This configuration insures that each origin set location and corresponding derivative set locations are unique to a particular constituent array. Referring to FIG. 4, the constituent arrays 415, 425, 445 each have sets including an origin set location and associated derivative set locations, the compositions of the locations designated by having the same alphanumeric code (letter case insensitive), that have more than one location that does not correspond with any other locations of any other origin set and its associated derivative set.

In another particular embodiment, two constituent arrays are configured as arrays with locations arranged in rows and columns, each constituent array having a common number of locations that are geometrically similarly positioned in each array. One constituent array, designated a X array, has an origin set of locations arranged in a vertical line, with each origin set location's corresponding derivative set configured in a horizontal line with one derivative set being adjacent to the origin set location; an example of which is depicted by array 415 in FIG. 4. The second constituent array, designated a Y array, has an origin set of locations arranged in a horizontal line, with each origin set location's corresponding derivative set configured in a vertical line with one derivative set being adjacent to the origin set location; an example of which is depicted by array 425 in FIG. 4. The arrays are combined in an assay array in a manner that preserves the orientation of the constituent compositions; an example of this is shown in FIG. 1 in which assay array 130 preserves the orientation of the constituent compositions from the constituent arrays 110 and 120 (e.g. combined composition 131 in the upper left hand corner of assay array 130 has constituent composition 116 and 126, both from the upper left hand corner of X array 110 and Y array 120, respectively).

Evaluating the Activity of Combined Compositions Having Three or More Candidate Entities

The embodiments of the inventions described earlier provide no limitation upon the number of entities that may be present in any candidate composition of a combined composition. In one use of the embodiments, each combined composition will be limited to having two or fewer candidate entities in order to minimize possible confusion regarding which entities are responsible for a change in state of a test entity. Constituent arrays, however, may be configured to enhance the ability to detect the activity in a combined composition having three or more candidate entities.

In an embodiment of the invention, the configurations of constituent arrays 415 and 425, depicted in FIG. 4, may be utilized to accelerate identification of entities that may produce activity in a combined composition. In these embodiments, typically three or more entities capable of affecting the activity of a test entity are present in each combined composition. The use of greater than pairwise entities in combined compositions may decrease the number of assays required to identify candidate entities capable of affecting the state of a test entity, thereby accruing the advantages of saved time and resources. As well, the embodiment may aid the identification of combinations of entities having unexpected interactions. Note that these embodiments may also be practiced with one or two candidate entities present in the assay array as well.

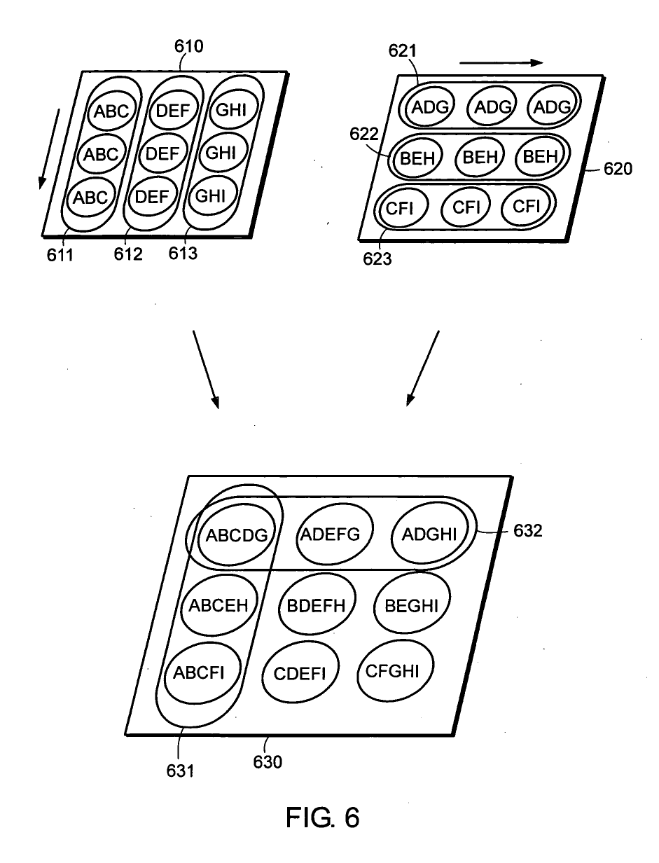

Referring to FIG. 6, an embodiment of the invention utilizes constituent arrays 610, 620, each containing constituent compositions having more than one entity potentially capable of affecting the state of a test entity, to produce an assay array 630. Every letter represents a candidate entity of a composition. For example, the locations 611 of array 610 each have a candidate composition with candidate entities A, B, and C.

Each location of an assay array holding a combined composition typically contains at least three candidate entities, though the embodiment may be used to test pairs of candidate entities, or even entities singularly, as well. Each constituent array contains a plurality of sets of locations. In the embodiment shown in FIG. 6, each location of a particular set contains the same constituent composition; other embodiments may not require this. Constituent compositions typically contain at least one candidate entity, though the number may vary set to set, and between constituent arrays. For example, one constituent array may utilize three entities in each constituent composition, while another constituent array utilizes two entities in each constituent composition. The quantity and concentration of entities in the particular set of locations may vary or be substantially identical. For example, the concentration of each entity in a set may be substantially identical and selected at an elevated concentration level to insure the triggering of a change in state of an evaluative composition. Each location in a constituent array has at least one corresponding location in every other constituent array. Furthermore, a plurality of locations in every set of locations having a particular constituent composition in a constituent array does not correspond to locations in any other set of locations with a given constituent composition in any other constituent array.

The constituent array configurations 610 and 620 of FIG. 6 illustrate one example of the above embodiment. Constituent array 610 holds sets of constituent compositions 611, 612, 613 in locations ordered in columns. Constituent array 620 holds sets of constituent compositions 621, 622, 623 in locations order in rows. Each location of a set of contains the same composition, each composition having a plurality of entities. Assay array 630 holds combined compositions in locations resulting from aliquots of constituent composition from the corresponding locations of the constituent arrays 610 and 620. The configuration of the sets of compositions in each constituent array 610, 620 is selected such that each combined composition in the assay array 630 does not have substantially the same composition.

Other embodiments of the invention include further modifications to the configuration of the constituent arrays that may aid the identification of entities that affect the activity of a combined composition. In a first modified embodiment, each entity utilized in a constituent array is also utilized on every other constituent array. Use of such embodiment helps create combined compositions that contain a given candidate entity in the presence of differing components of a composition. As one example shown in FIG. 6, entity A is utilized in set 611 of constituent array 610 and set 621 of constituent array 620. Assay array 630 incorporates entity A in locations denoted by sets 631 and 632. Set 631 includes compositions that include entity A, but always in the presence of entities B and C. Utilizing entity A in constituent array 620 allows combined compositions to be formed in assay array 630 that have entity A without the presence of entities B and C. Thus any effects in activity due to the collective behavior of entities A, B, and C in combination may be discerned.

In a second modified embodiment, any composition utilized in a set of locations of a constituent array is not utilized in any other set of locations in any constituent array; thus each set of combined composition locations has a combined composition that is unique. Such an embodiment aids in minimizing overlapping compositions in combined compositions of an assay array, and helping insure the uniqueness of combined compositions that are produced. As one example in FIG. 6, each set of locations 611, 612, 613, 621, 622, 623 in the constituent arrays 610, 620 has a unique composition which is not repeated in any other set.

In a third modified embodiment, each entity of a particular composition, used in a set of locations in a constituent array having the particular composition, is not utilized with any other entity of that same composition in any other locations of any constituent array. This embodiment, like the second modified embodiment, helps insure the uniqueness of combined compositions that are produced. The configuration of the arrays in FIG. 6 provides an illustration of the embodiment.

Quality Control of Assay Array Data

Evaluation of combined compositions may be facilitated by the use of composition controls in an array. In an embodiment of the invention, a composition control set of locations is assigned to each constituent array. When each location of each array has at least one corresponding location in every other array, the locations of the composition control set of a constituent array are chosen such that they do not overlap with a corresponding location in any other constituent array that contains a constituent composition or any control.

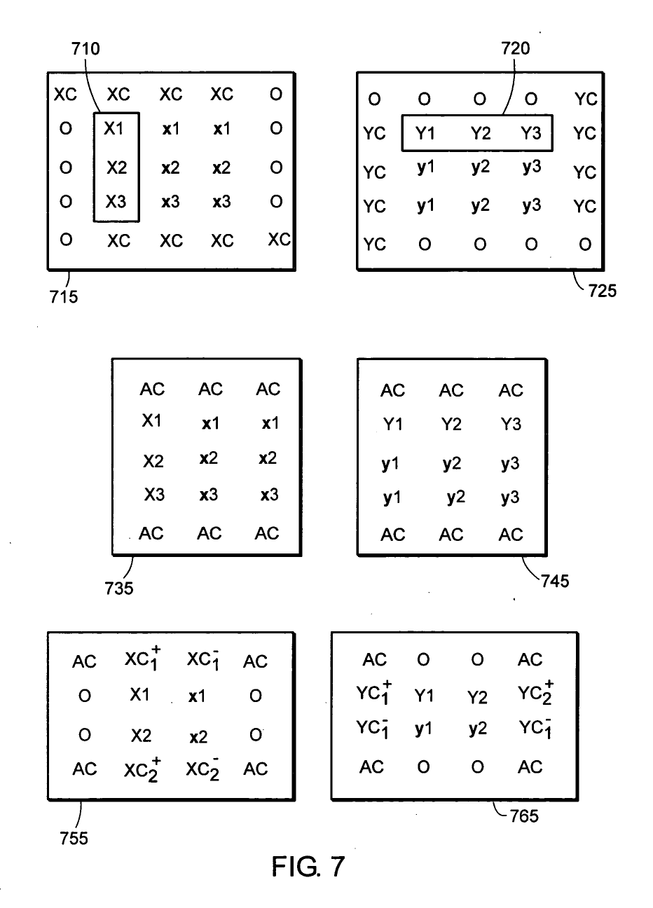

Arrays 715 and 725 of FIG. 7 illustrate diagrammatically an embodiment of two constituent arrays with locations that incorporate control compositions. Array 715 represents a constituent array, with an origin set of locations 710 and each origin location's corresponding derivative set arranged in a horizontal row. The label XC represents locations having a composition control associated with the constituent compositions of the X constituent array 715. Array 725 represents a constituent array, with an origin set of locations 620 and each origin location's corresponding derivative set arranged in vertical columns. The label YC represents locations having a composition control associated with the Y constituent array 725. The symbol O indicates an empty location in the constituent arrays 715 and 725.

When constituent arrays utilizing composition controls are combined to form an assay array, composition controls may provide a number of advantages for evaluating the activity of combined compositions. In one instance, the presence of an empty location in the assay array corresponding to a composition control location of a given constituent array may serve as an indictor that the constituent compositions associated with the given constituent array have not been added to the assay array. This may be particularly of use in a process in which automated equipment has malfunctioned and a user cannot determine the state of a given assay array's contents.

In another instance, the contents of the composition controls of each constituent array in an assay array may be used to help determine the quality of data in an assay array, i.e. whether the combined composition of an assay array has been contaminated or subject to an environment affecting the activity of the composition (sometimes referred to herein as quality control). Though the evaluated activity of a given composition control has an expected quantity, the actual measured value of the activity will naturally vary depending upon the random error associated with the measurement and possible systematic errors introduced to the assay array from combining compositions or other processes associated with the assay array. Statistical analysis of the measured values of the control compositions may provide an indication of the possible error introduced in an assay array. Measures are chosen in an attempt to maximize the possible use of data while minimizing the possible occurrences of false positive and false negative errors from an assay array. The measures may also help manage the time of researchers by providing an indication of whether assay arrays contain acceptable or unacceptable data, or should be further scrutinized manually to determine the data's acceptability.

One method of estimating possible errors introduced to an assay array is to calculate a z-factor based upon the measured values in the locations corresponding to constituent controls. Positive and negative controls are utilized, each having an expected activity value, respectively. Measured values of activity for all control locations are taken, with an average and standard deviation calculated for the positive controls (μ₊ and σ₊, respectively) and negative controls (μ₋ and σ₋, respectively). The z-factor is then calculated using the equation:

$$z = 1 - \frac{3 ( {σ}_{+} + {σ}_{-} )}{{μ}_{+} - {μ}_{-}}$$

The average values, μ₊ and μ₋, may utilize either a numerical average or a median average based upon all the measured positive and negative control values respectively.

To the extent that systematic errors may be introduced when creating an assay array, the z-factor may provide a measure of the presence of such errors. When the calculated value of z is close to 1, the z-factor indicates the spread of the data is small relative to the average value, which may indicate that the errors present are relatively small. Conversely, the errors in identifying control values may be substantial when the value of z is much smaller than one, indicating that substantial variation is present in the expected control values.

In an embodiment of the invention, the z-factor is used to decide whether data from an assay array is of sufficient quality to be acceptable. If the z-factor is above a value Z_{above}, the data from an assay array is considered of acceptable quality. If the z-factor is below a value Z_{below}, the quality of the data from an assay array is considered unacceptable; the data is not utilized and another assay array may be prepared to obtain acceptable data. If the z-factor lies between Z_{above} and Z_{below}, the data on the assay array is examined manually to determine the data's quality. In a particular embodiment, Z_{above} is chosen to be substantially between 0.6 and 0.7, while Z_{below} is approximately 0.4.

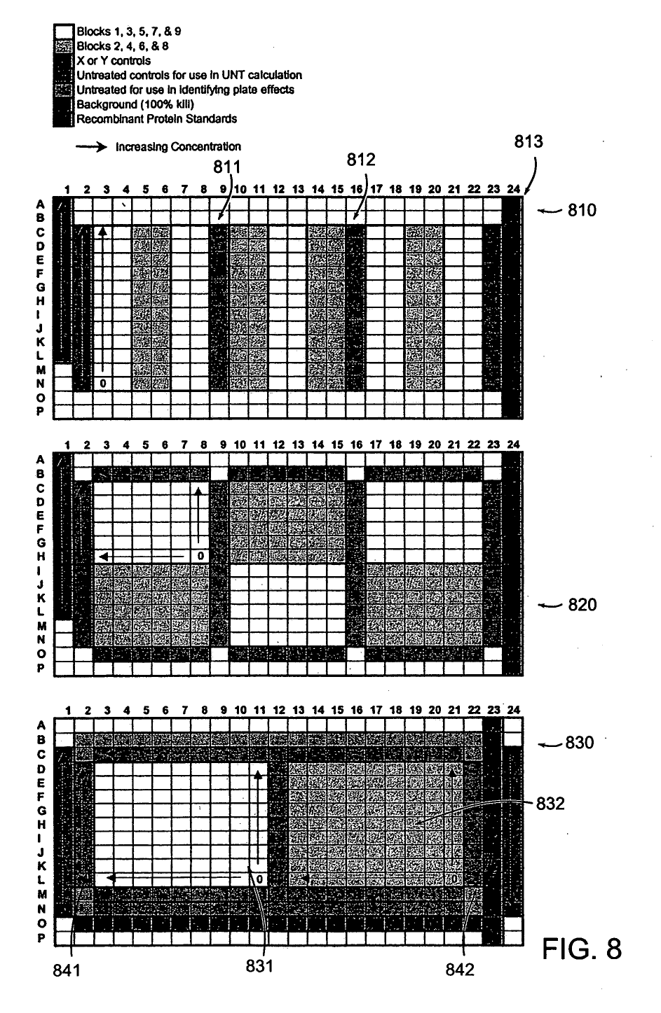

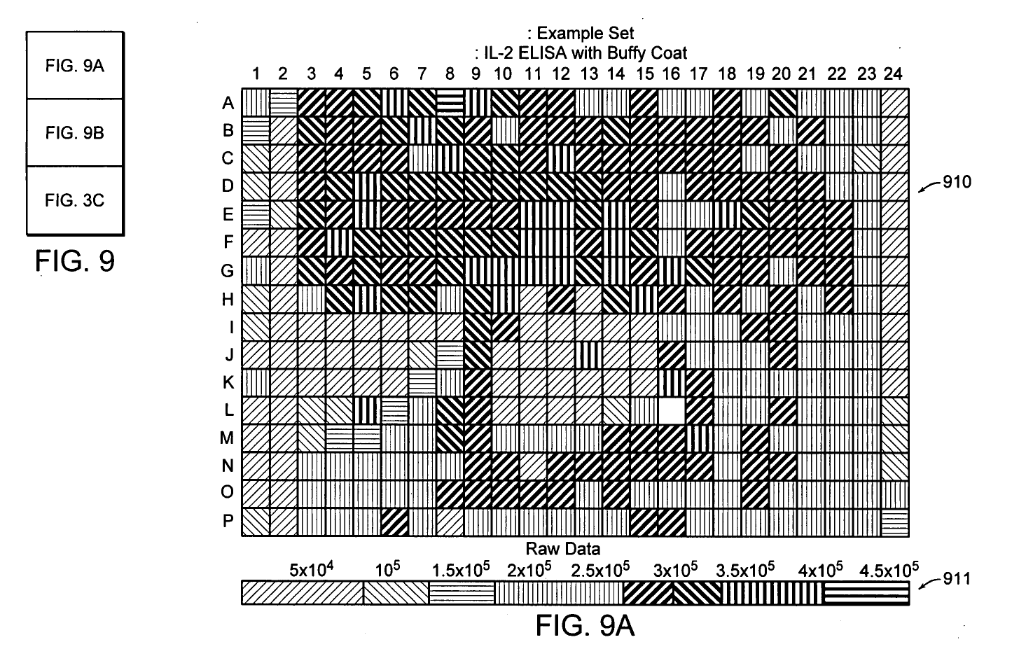

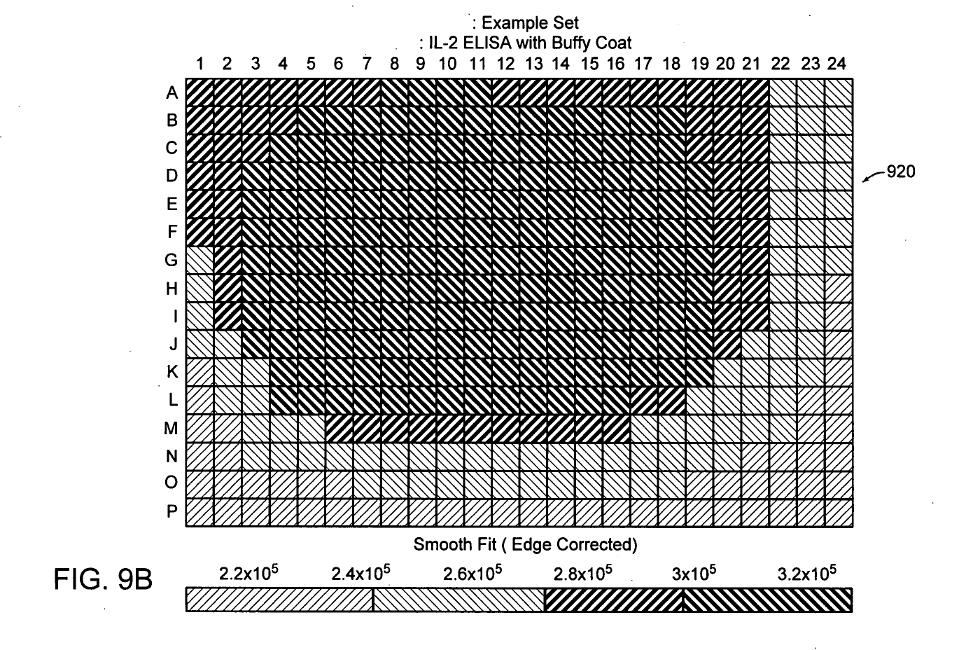

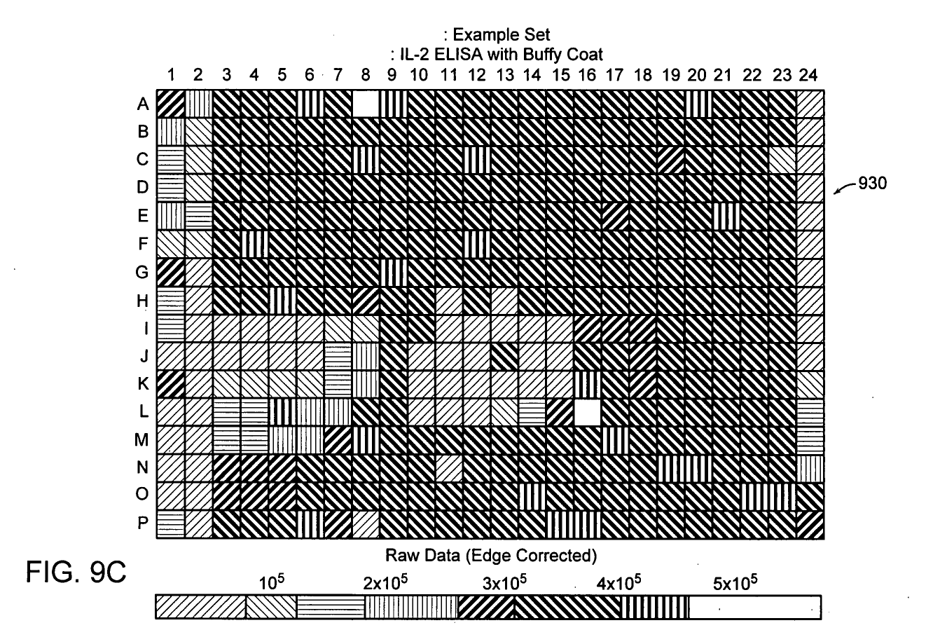

Another method of estimating possible errors relies upon a measure known as a global c-value. The global c-value is utilized when separate blocks of locations are utilized on a physically distinct object of an assay array, as diagrammatically illustrated in FIG. 9. Each block is associated with a set of positive controls that are serially diluted from a highest to a lowest concentration. For example, assay array 830 in FIG. 8 contains two 9×9 blocks of locations 831, 832 holding combined compositions, each block associated with a block of positive controls 841 and 842. For each location of highest concentration of control associated with each block, a local “quantized” c-value is assigned depending upon the quotient, Q, of the measured activity in the highest concentration control location divided by a normalization value; the local c-value is quantized in that the value may only be assigned one of a finite number of possible values.

In one particular embodiment, if the quotient is above a value Q_{above}, the assigned local quantized c-value is C_{high}. If the quotient is between Q_{above} and Q_{below}, the assigned local quantized c-value is Cᵢₙₜ. If the quotient is below Q_{below}, the assigned local quantized c-value is C_{low}. All local quantized c-values from each block of a physically distinct object of an assay array are numerically averaged to determine a global c-value for the physically distinct object of the assay array. Depending upon the value of the global c-value, a determination may be made as to whether the data from a particular assay array is of acceptable quality. The values of Q_{above}, Q_{below}, C_{high}, Cᵢₙₜ, and C_{low} may be chosen in any manner suitable to the attain the specific level of quality control desired by a user. In a particular embodiment, Q_{above} may have a value substantially between 0.7 and 0.8, while Q_{below} has a value of approximately 0.6. In another particular embodiment, the values of C_{high}, Cᵢₙₜ, and C_{low} are 1, 0.5 and 0, respectively. Other embodiments may utilize different specific values for Q_{above}, Q_{below}, C_{high}, Cᵢₙₜ, and C_{low}, or utilized a different number of possible values for C, setting appropriate limits for Q to transition between the various C values.

Embodiments of the invention utilizing the global c-value may use any normalization value of convenience. One normalization value that may be used is based upon the measured activity in a well with a compound having an expected activity level of zero with respect to some test entity. Another normalization value that may be used is based upon a measured activity level in a location where no test entity is present, i.e. a background measurement. A third normalization value that may be used is to assume that the activity level has a specific value. Any of these normalization values, among others, may be utilized to determine Q.

As is apparent to those skilled in the art, Q need not be a normalized value but can be based upon some other scale of activity measurement.

Other methods of implementing quality control measures for assay arrays may include evaluating the activity of compositions in the constituent control locations of an assay array in which a control composition is serially diluted. Comparison of the measured activity in the wells with an expected activity in the wells may also provide a measure of error that may be present in an assay array. Constituent control wells of an assay array may also contain a serial dilution of a specific candidate composition associated with a particular constituent composition. Again, comparison of the measured activity due to a candidate composition from a constituent composition may be compared with the expected response in order to provide a measure of possible error in the assay array. Comparison techniques may include comparing an average value from a set of measurements, or some type of functional comparison of a response vs. concentration curve. In general, application of statistical analysis techniques in comparing one or more measured control values with expected control values may provide a method of measuring the data quality of an assay array.

Accurate evaluation of the assay array may also be facilitated by the use of an assay control to help identify and correct any errors in evaluating the activity determined from a plurality of locations in an assay array. An assay control comprises a substance with a known activity in an assay array. The assay control may also be present in the constituent arrays that are combined to form the assay array, the assay controls added to the assay array from the constituent arrays. Alternatively, the assay controls may be added to the assay array by direct transfer from one or more source containers having the assay control. The set of locations in an assay array that hold an assay control have corresponding locations in each constituent array, the corresponding locations of the constituent array not having a composition or a composition control. Arrays 735 and 745 illustrate the locations of the corresponding locations of assay controls, designated by the label AC, in a constituent array; these locations may either contain the assay control or be empty in accordance with either of the two methods for adding assay controls described above.

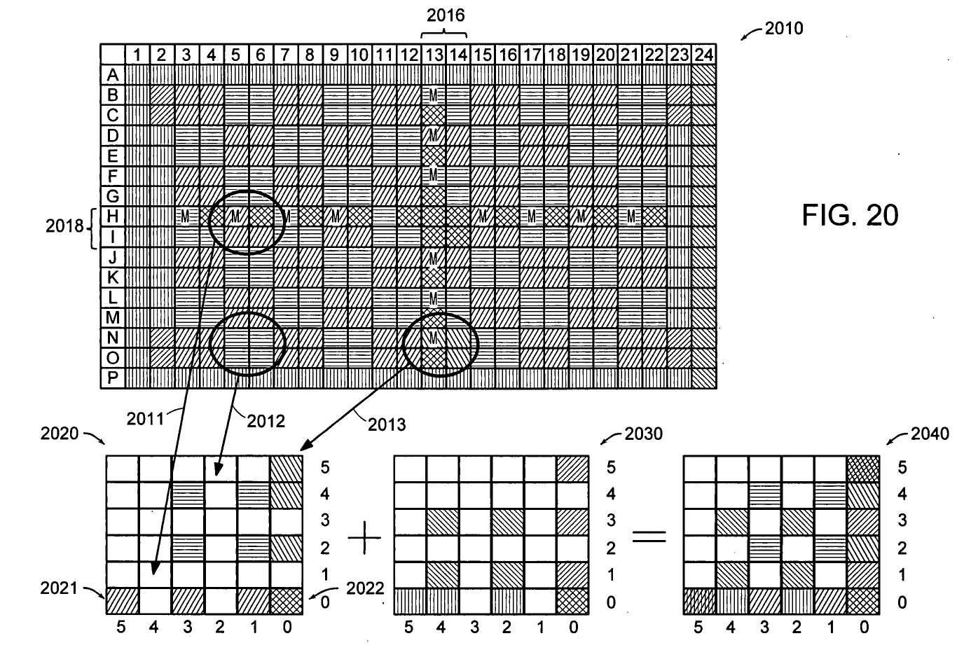

Assay controls may enable the correction of systemic error in data associated with evaluating a combined composition in an assay array. For example, when arrays are embodied as plates with wells, wells located near the edge of a plate may be subject to greater temperature variations and other environmental changes relative to well locations in the middle of a plate. In such instances, controls in wells close to an edge may not be measured with an activity that matches the expected value. The deviation of the measured values in an assay array from their expected values may provide an offset correction at specific locations of the plate, or provide a general mapping of offset correction as a function of location throughout a plate. This deviation may be used to apply a correction to all other locations of an assay array. The deviations may be calculated by any means known in the art of data correction including fitting a function that predicts deviation as a function of location, and applying that deviation to correct the data. Thus an embodiment of the invention includes distributing assay controls in various places throughout an array, including at least one location near the edge of a physically distinct object that constitutes a portion, or in a pattern from one end of the array to another, as depicted by the array 2010 in FIG. 20.

FIG. 9 illustrates diagrammatically an example of using assay controls to correct for edge effects in an assay array. The array 910 depicts the values of evaluated activity in each location of a 386 well plate; the color of each cell corresponding to an activity level as indicated by the key 911 shown as the bottom row of the array 910. The locations marked by O in FIG. 9 represent locations containing an assay control utilized to account for edge effects. Array 920 provides values of “evaluated activity” based upon a functional fit of the measured values of activity utilizing the locations containing an assay control. The values of each location in array 930 are the result of dividing each location of array 910 by the value in the corresponding location of array 920, array 930 providing a corrected set of values for the activity of the combined compositions.

In a preferred embodiment of the invention, assay controls and composition controls are incorporated into a constituent array and assay array simultaneously. In such a preferred embodiment, each constituent array and assay array has at least 4 locations: one location holding a composition in a constituent array or a combined composition in an assay array; one location corresponding to an assay control; and two locations corresponding to constituent controls, one location for each constituent composition. Arrays 755 and 765 of FIG. 7 illustrate diagrammatically another embodiment of configurations of constituent arrays, with assay control locations (AC) and composition control locations (XCᵢ⁺, XCᵢ⁻, YCᵢ⁺, YCᵢ⁻) depicted, where i=1,2 to denote a specific composition control; + corresponds to a positive control location, and − corresponds to a negative control location. Combining the constituent arrays 310, 320 to form combined compositions on an assay array 330 is shown in FIG. 3, wherein locations corresponding to assay controls and constituent controls are depicted using the same notation as used in FIG. 7. Specific configurations of an assay array as embodied by 384 well-plate are shown in FIG. 8. Array 810 of FIG. 8 depicts a configuration utilizing 9 possible blocks of wells arranged in a 2×12 matrix for combined compositions. Array 820 depicts a configuration utilizing 6 possible blocks of wells arranged in a 6×6 matrix. Array 830 depicts a configuration utilizing 2 possible blocks of wells arranged in a 9×9 configuration. Locations for wells containing assay controls (labeled ‘untreated’), constituent controls (labeled ‘X or Y controls’), and material for determining a normalization value (labeled ‘background’) are also depicted in each configuration.

Analysis of Evaluated Activities of Combined Compositions

In the context of drug discovery, use of the aforementioned embodiments of the invention may facilitate identification and analysis of novel candidate compositions by providing an ordered configuration for the evaluated combined compositions. In particular, embodiments of constituent arrays 410 and 420 as depicted in FIG. 4, including the use of serial dilution in the derivative sets and the use of constituent controls and assay controls, allow for normalization of evaluated activities that may aid the identification of novel candidate compositions and analysis of the quantities of entities of the compositions that exhibit combination effects.

Referring again to array 910 of FIG. 9, where the arrays are embodied as plates with wells, the absolute evaluated activity in each well, as indicated by a measured value constituting raw data, is a function of a variety of variables that may include the type of testing performed, any errors introduced due to measurement and plate handling, background readings of the instrument, and the activity due to the interaction of a candidate composition with a test entity. In order to provide a standard measure of activity, independent of the type of test utilized or background reading, raw data may be normalized.

Normalization involves conversion of the data to provide a consistent numerical basis for the values of the converted data. For example, if a combined composition is sought to suppress the presence of a particular cell product, a candidate composition may be mixed with the particular cell product and tested for the presence of the product, less product corresponding to a more active candidate composition. Thus, the measured values may be normalized in a quantity known as inhibition:

$$I = 1 - \frac{m}{U}$$

where I is the inhibition; m is the measured value of activity; and U is an untreated location, which is the measured value of activity in a location not exposed to the candidate composition.

Theoretically, I may take values ranging from one to zero, I=1 when a candidate composition completely suppresses the presence of the cell product since m=0 in that instance, and I=0 when a candidate composition has no effect on the presence of a given product since m=U. In reality, the presence of random error causes measurements associated with m and U to fluctuate from their expected values; thus I may deviate from staying within the range of one to zero.

In instances where a background signal from an evaluation technique is present, even when no suppression of a cell product has taken place, the background signal may be accounted for by subtracting the background signal, B, from both the measured value of activity, m, and the measured value in an untreated location, U, and substituting these values for m and U in the inhibition calculation. B may be obtained in manner known to those skilled in the art of the particular evaluation technique; for example B may constitute a measured activity in a well with no test entity.

In order to reduce the effects of random error, measurements of activity in several locations for U and B may be performed. Thus an average value for the measured activities of the untreated locations, U, and background locations, B, may be calculated. These average values may then be utilized to calculate the inhibition where a measured activity, m, replaced with the value of m−B, and the activity in an untreated location U, is replaced with the value of U−B.

As described earlier, composition controls and assay controls may be utilized for quality control determinations of particular physical embodiments of arrays. The controls, however, may also be utilized in the normalization of data. Values for U or U may be based upon the evaluated activity in one or more locations corresponding to having a negative composition control. In the context of inhibition, a negative composition control does not suppress the presence of the cell product. U may utilize measurements in 10-30 locations in order to obtain a statistically satisfactory value. For example, columns 811 and 812 of array 810 in FIG. 8 may be used to calculate U for the data contained in the 2×12 blocks of the array. As well, an ideal background reading corresponds to a situation where the cell product is completely suppressed; no activity is detected with the exception of what is expected as a background reading of instrument. Several types of assumption and measurements may be utilized to provide a particular basis for B. Three different, but useful, bases for B include: (i) using the measured activity in one or more wells that have an expected activity level of zero (e.g. one or more wells containing a positive constituent control or a substance with a very high probability of suppressing the measured activity); (ii) using the measured activity in one or more wells that has no test entity present, any signal generated thus corresponding to background (in the current example, a measurement is made in a well without the cell product); and (iii) a priori assuming the average background reading is zero. With regard to methods (i) and (ii), in an embodiment of the invention, wells of a plate may be reserved for these measurements. For example, in FIG. 8, measurements in the locations of column 813 may be utilized to calculate B. Method (iii) has the advantage of assuring that noise will not be introduced into values of L Locations containing an assay control may also be utilized as wells for determining U, U, and B, assuming they hold an appropriate composition.

I provides a unitless measure of the inhibition that is independent of the type of measurement utilized to determine activity since the signal associated with a particular measurement is scaled relative to the corresponding untreated signal. Providing measurements of evaluated activity in terms of inhibition may aid in the comparison of data sets utilizing comparable entities as candidate compositions. For example, if two identically prepared combined compositions are tested for an evaluated activity on different days, one combined composition may have systematically higher values due to some change in instrumentation reading causing a change in background signal. Viewing the data for each combined composition in terms of inhibition reduces such systematic error. Viewing data in terms of inhibition may also allow comparison of data detected by two different methods, e.g., testing the same candidate compositions using different test entities. Though the raw data of each measurement differs because the detection mechanism differs, conversion of the data sets into unitless inhibition may allow for easier comparisons of the data sets.

Identification of candidate compositions that induce a combination effect may be enhanced by examining the difference between the measured activity of a candidate composition and a predicted value from a model that utilizes the measured activity of one or more of the components of the candidate composition, providing some indication of how the components act independently. It may be convenient to present the difference values in terms of a difference in inhibition between the measured value and predicted value, as described in the examples herein. Any model that provides some measure of the individual entities' expected activity may be utilized. Some particular models are described herein.

In one model, the measured activity in terms of inhibition is compared to the inhibition response of the highest single agent of the candidate composition. For example, if a candidate composition is composed of entity A at concentration C_{A} that produces an activity level I_{A} when independently exposed to a test entity, and entity B at concentration C_{B}, that produces an activity level I_{B} when independently exposed to the test entity, the greater of I_{A} and I_{B} is used to calculate the difference.

In a second model, the measured inhibition is compared to the predicted inhibition of the candidate composition if the candidate entities interacted according to the Bliss Independence Model. For a candidate composition as noted in the above example, the Bliss Independence Model states that the predicted inhibition, I_{BI}, will have the form:

I_{BI}=I_{A}+I_{B}−I_{A}I_{B}

The term I_{A}I_{B} is subtracted off to account for the statistical competition between entity A and entity B.

In a third model, the Loewe Additivity Model, the measured inhibition is compared to the predicted inhibition at a concentration of entity A equal to C_{A} and concentration of entity B equal to C_{B} that satisfies Loewe's self-replacement criteria:

$$\frac{{C}_{A}}{{C}_{A} \vert {I}_{A} = {I}_{LA}} + \frac{{C}_{B}}{{C}_{B} \vert {I}_{B} = {I}_{LA}} = 1$$

where Cᵢ|Iᵢ=I_{LA} is the concentration of entity i such that the inhibition of the single entity i is equal to the value I_{LA}. Thus for a given candidate composition composed of entities A and B and concentration C_{A} and C_{B}, the inhibition predicted by the Loewe Additivity Model is the inhibition I_{LA} that satisfies the above equation. Since the equation cannot be solved algebraically, various root-solving methods known to those skilled in the art may be employed to solve implicitly for I_{LA}.

Conversion of the evaluated activity of combined compositions from data readings to values of inhibition, and calculations to compare inhibition values based on the evaluated activities with predicted inhibitions based on a model of how individual entities are expected to behave, may be achieved by any means known to those in the art of data conversion and computation. For example software packages such as CalculSyn (BioSoft, Ferguson, Mo.), which calculates a standard dose effect and synergy model based on the methods of Chou and Talalay, and CombiTool (Biocomputing, Institute of Molecular Biotechnology Postfach 100813, D-07708, Jena Germany), which calculates a Loewe Additivity Surface, allow users to compare observed data with predicted values based on a model. Alternatively, such calculations may be performed using standard spreadsheet and computational software, such as Microsoft Excel (Microsoft Corp., Redmond, Wash.) and Microsoft Visual Fox Pro (Microsoft Corp., Redmond, Wash.), may be custom-coded to perform the necessary calculations.

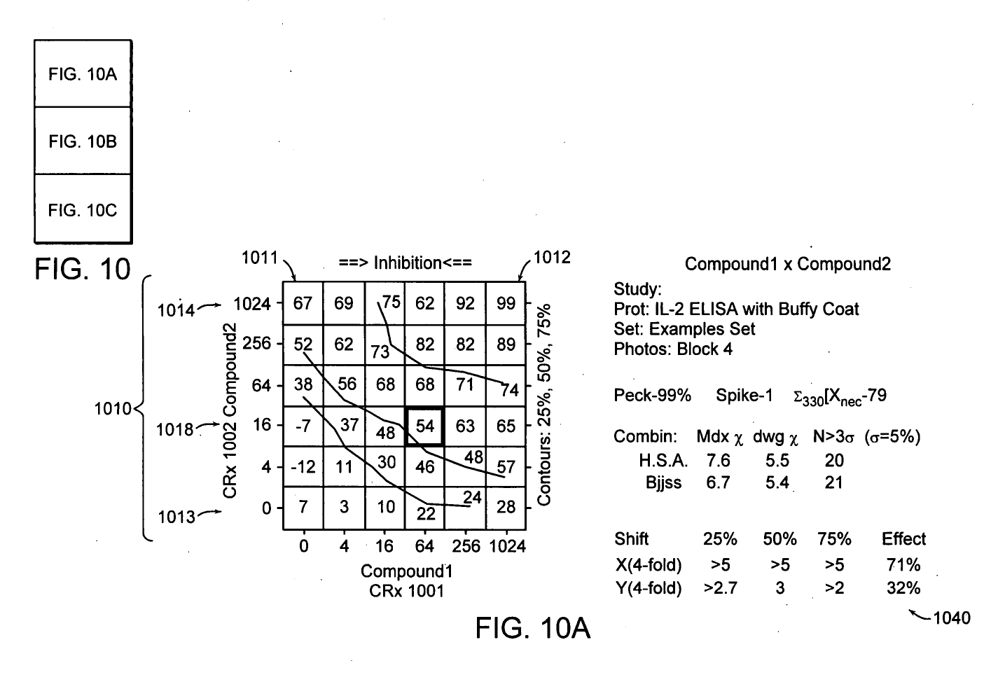

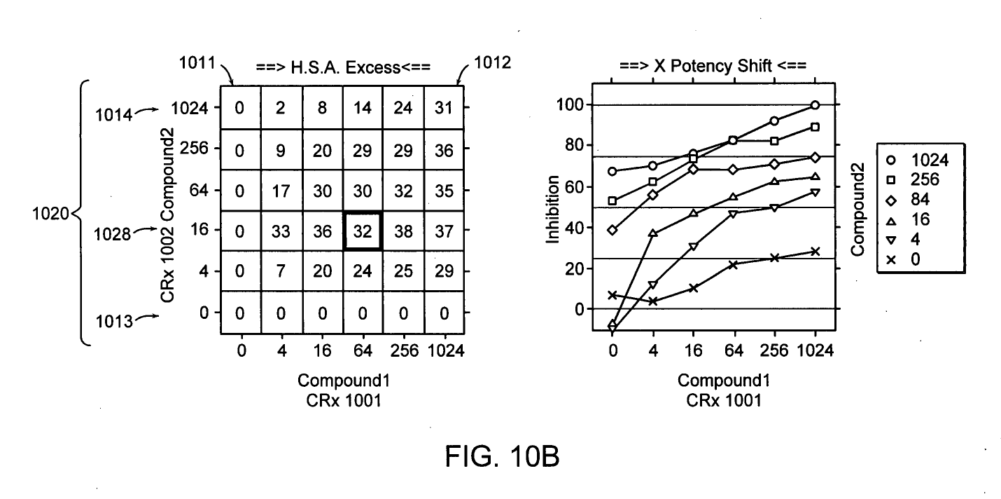

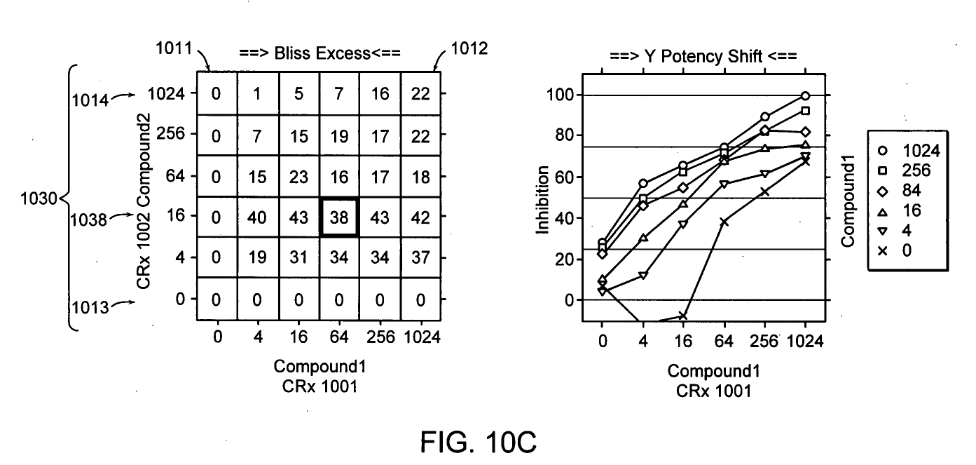

As mentioned earlier, formation of an assay array using constituent arrays 410 and 420 configured as depicted in FIG. 4 with serial dilutions, along with viewing the evaluated activity in each location in terms of inhibition and the difference of the inhibition relative to a model representing the entities acting independently, may enhance the identification and evaluation of potentially attractive combined compositions. Referring to FIG. 10, matrices 1010, 1020, 1030 represent the same data obtained from a 6×6 assay array holding 36 combined compositions including a candidate composition consisting of two components. Specifically, component 1 has a concentration that increases in steps of a factor of four relative to some base concentration, proceeding in wells that move from left to right. Thus, the wells in column 1011 contain a concentration of component 1 of zero, while the wells in column 1012 contain a concentration of component 1 equal to 1024 times the base concentration. The wells in row 1013 contain a concentration of component 2 of zero, while the wells in row 1014 contain a concentration of component 2 equal to 1024 times the base concentration. Note that the wells of column 1011 and row 1013 provide data for calculating the inhibition of the individual candidate entities compound 2 and compound 1, respectively, at the various concentrations utilized in the array because of the absence one of the candidate entities; the data in these locations provide values required in the aforementioned predictive models for comparison with the measured values. The layout of serial dilutions of the two components is enabled by the earlier described embodiments as depicted in FIGS. 3A and 3B.

Matrix 1010 presents measured inhibition values at each location of the assay array. The normalized inhibition is presented in each location on a percent basis, and color-coated according to the location's value in reference to the color-coating key 1040. The stepwise changes in concentration in the horizontal and vertical directions, corresponding to concentration changes for a particular component depending upon the direction, enable a two-dimensional functional representation of how inhibition changes as a function of candidate composition concentration, i.e. a function of the concentration of compound 1 and compound 2. As well, the systematic change in concentration may facilitate the interpolation and extrapolation of evaluated activity beyond the actual combined compositions measured. For example, the systematic layout of concentrations in matrix 1010 allows a depiction of iso-inhibition contours 1015, 1016, 1017, each graph representing a set of concentrations that produce an inhibition of 75%, 50%, and 25%, respectively, according to the measured activity of the combined compositions. Such graphical representations may enable identification of critical concentrations in relation to a desired threshold of inhibition.

In addition, the configuration of wells in terms of systematic concentration changes also may facilitate the identification and removal of evaluated activity locations that contain erroneous values; this process is known as spike filtering. Since concentrations of each entity of a candidate composition are systematically distributed, locations with clearly erroneous values of activity may be readily identified; these locations are known as spikes.

Erroneous values of activity may be identified by any method known in the art. For example, in some instances the values may be readily identified by manual inspection of the data. In another example, a plurality of the measured values of activity in an assay array are extrapolated or interpolated to provide model values of the evaluated activity at the combined concentrations. Erroneous measured values of evaluated activity in an assay array may then be identified when the difference of a model value and measure value in a given location exceeds a particular threshold value. This threshold value may also be based upon adjacent values of evaluated activity not exceeding a threshold concentration gradient.

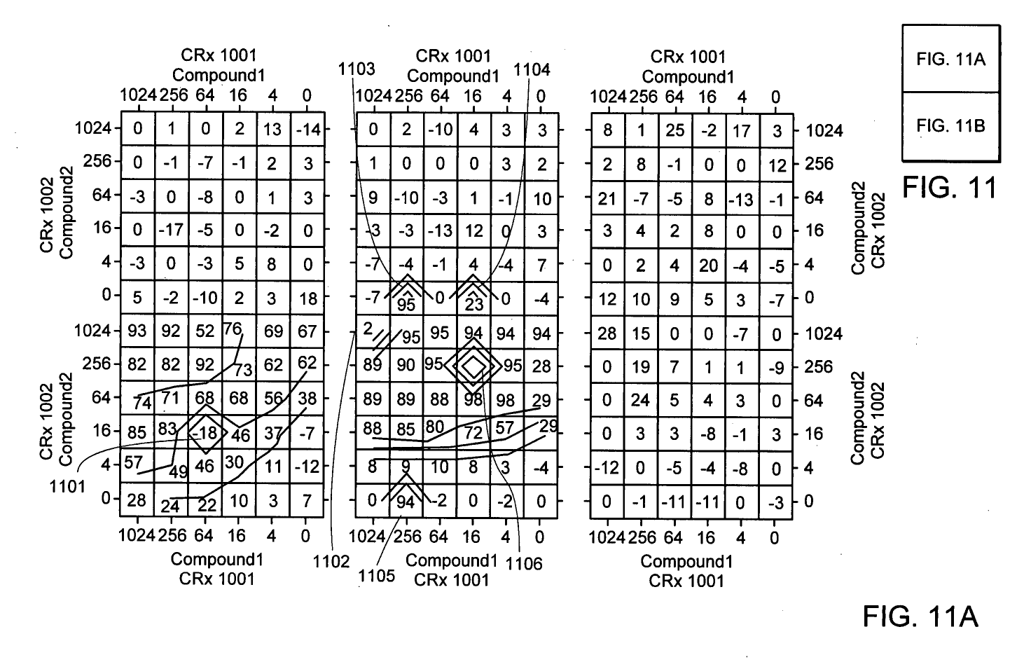

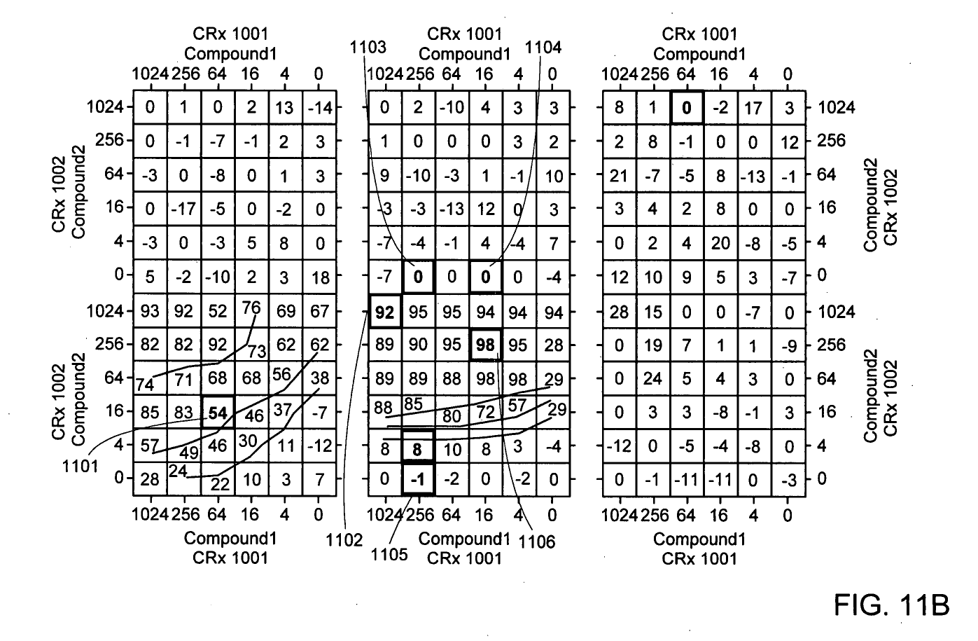

The activity originally assigned to a spike may be replaced by assigning a value consistent with values accorded to the neighboring locations in order to obtain a smooth monotonically changing surface. Any relevant method known in the art of data analysis may be utilized to obtain the new values in a spike. Example of methods include using the median of the values assigned to adjacent locations to the spike, or fitting a functional surface using the data of the neighboring locations and determining the value at the spike from the fitted function. Thus the replacement values may depend upon either or both of the location concentration of one or more entities around the location value to be replaced, and one or more values of activity adjacent to the location value to be replaced. FIGS. 11A and 11B provide an illustration of the removal of spikes in locations 1101, 1102, 1103, 1104, 1105, and 1106, FIG. 11A depicting values of the inhibition before spike filtering and FIG. 11B providing values of inhibition after the spike filtering.

Matrices 1020 and 1030 in FIG. 10 present calculated values of the difference between the measured inhibition and the predicted inhibition according to the highest single agent model and the Bliss Independence Model, respectively. Row 1013 and column 1011 provide the individual candidate entity inhibitions for use with the predicted models. Again, the concentration of components 1 and 2 are represented in the corresponding positions as described for matrix 1010, each location having a value corresponding to the difference between the measured inhibition and the predicted inhibition on a percent basis. Viewing the evaluated activity in terms of calculations presented by matrices 1020 and 1030, as a systematic function of concentration of the individual entities, as enabled by the embodiments of the invention, may allow improved identification of candidate compositions that present synergistic properties at particular concentrations of the entities. For example, matrix 1010 shows steadily increasing inhibition as the concentrations of component 1 and component 2 is increased. Since each individual component is expected to result in increased inhibition as the component's concentration is increased, as shown by 1011 and 1013, identifying precise concentrations of each component that have a synergistic combination may be difficult by briefly observing matrix 1010. From matrices 1020 and 1030, however, synergistic combinations may be identified by locations with high numerical values since an expected inhibition of the components as predicted by a model, is subtracted off. In particular, the row 1018, 1028, 1038 corresponding to a concentration of compound 2 at 16 times its base concentration seems to have particular synergistic inhibition in the presence of compound 1 as depicted by the values in rows 1028, 1038. The synergy is not as easily identified by looking at row 1018 of matrix 1010.

Though the discussion in the preceding paragraph is provided in the context of identifying synergistic effects, the difference value matrices may be used to aid identification of any type of combination effect.

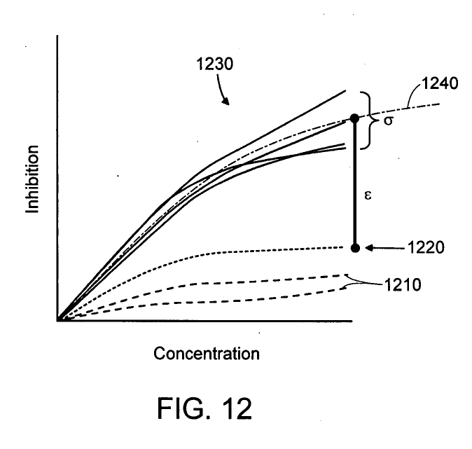

Embodiments of the invention may enhance the ability to identify synergistic combinations by allowing repeated evaluation of a range of concentrations to insure that identified synergistic combinations are not the result of errors in data. Referring to FIG. 12, for a given set of combined compositions a plot of inhibition as a function of concentration may be created. Random and systematic errors, however, may result in incorrect identification. Thus, evaluating the activity of the combined composition using multiple trials may produce a composite result with better accuracy than expected from a single trial. As shown by array 820 of FIG. 8, since multiple blocks may be utilized on a plate, each block may be designed to contain the same combined composition in order to obtain multiple trials of the same combined composition. Alternatively, a given assay array may be recreated multiple times and evaluated (e.g. utilizing the embodiments of FIG. 3 or FIG. 5).

The data from each trial may be utilized to create a representation of inhibition vs. concentration of the combined composition. In FIG. 12, a one-dimensional representation of inhibition vs. concentration graphs for a number of trials 1230 is shown, having some representative spread in value, a, for each value of concentration (e.g. standard error). An average inhibition vs. concentration profile 1240 may be calculated by averaging the profiles 1230 of each trial. The difference, ε, between the average inhibition and the expected inhibition based upon some expectation model, such as highest single agent 1210 or Bliss Independence 1220, may be used as a measure of synergy as discussed earlier. However, when the spread of inhibition σ is large relative to the difference value ε, the difference value alone may not provide good representation of synergy. Therefore, other measures that account for the deviation may provide a better representation. For example, using a measure of ε/σ in place of ε may allow identification of combinations that are particularly potent since large values of ε/σ indicate that the measured difference is large relative to spread in the data.

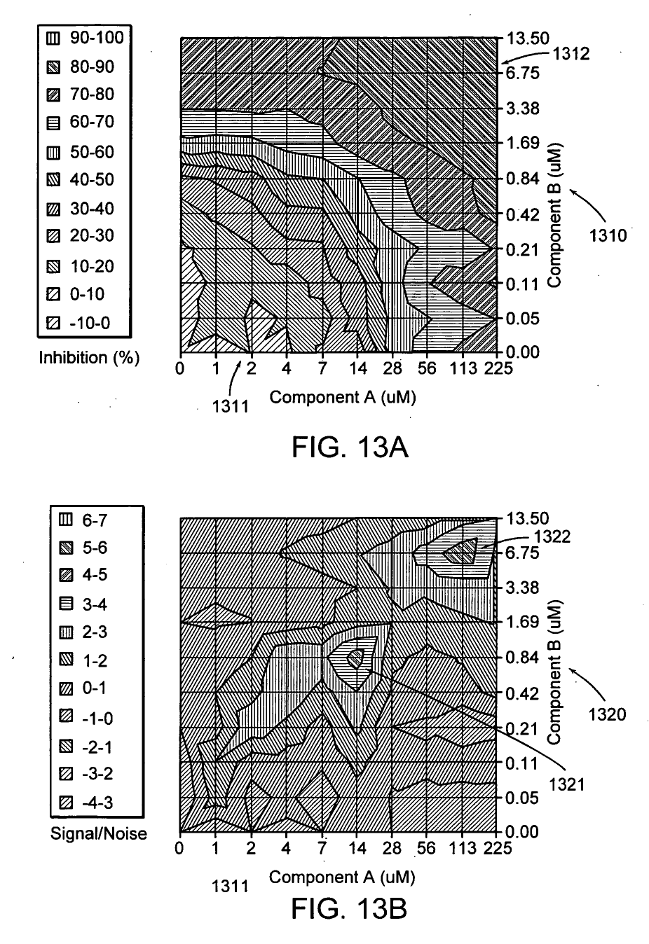

Referring to FIG. 13, matrix 1310 depicts data from a 10×10 assay array in which values of inhibition for various locations are plotted using color to denote the inhibition value, each location having a corresponding concentration of component A and B relative to some base concentration as depicted along the axes, 1311 and 1312. The same data are used to calculate ε/σ relative to a highest single agent model; the values of ε/σ are represented on matrix 1320. The peak value regions 1321 and 1322 shown in matrix 1320, identify potential candidate compositions at specific concentrations of entities which may provide especially synergistic inhibition; the regions are not identified as easily by viewing matrix 1310.

Alternatively, σ may be used as an estimate of the uncertainty in values of ε. Thus plots of ε as a function of location are assessed along with local values of σ to provide a measure of the quality of the values of ε.

Identification of synergistic or antagonistic candidate compositions may be performed by manual inspection of the inhibition and difference plots herein described. Alternatively, automated methods utilizing data analysis methods known to those in the art may be employed. Methods may search for particular values above or below a critical threshold, or employ image analysis techniques wherein the data are represented by a contour plot, to name two non-limiting examples.

The facilitation of identification of synergistic combinations of candidate compositions by the above-described embodiments may also allow the development of a measure of synergy associated with a block, a physically distinct object, or an entire assay array based upon values associated with synergy (e.g. difference of inhibition from an model predicted inhibition, or the ratio of the aforementioned difference to the deviation in measured inhibition). Statistical analytical methods known to those in the art may readily be applied to provide these measures. For example, a measure of the “synergy” in an array may utilize the sum of a set of values of ε over a plurality of locations of the array, and the square-root of the sum of σ² for the plurality as a measure of error. These measures may be utilized to help users identify arrays or portions of array which should be analyzed manually for synergy.

Embodiments of the invention that may facilitate evaluation of combined compositions through identification and analysis of activities associated with an assay array may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over a network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Methods of Enhancing Activity Identification Efficiency

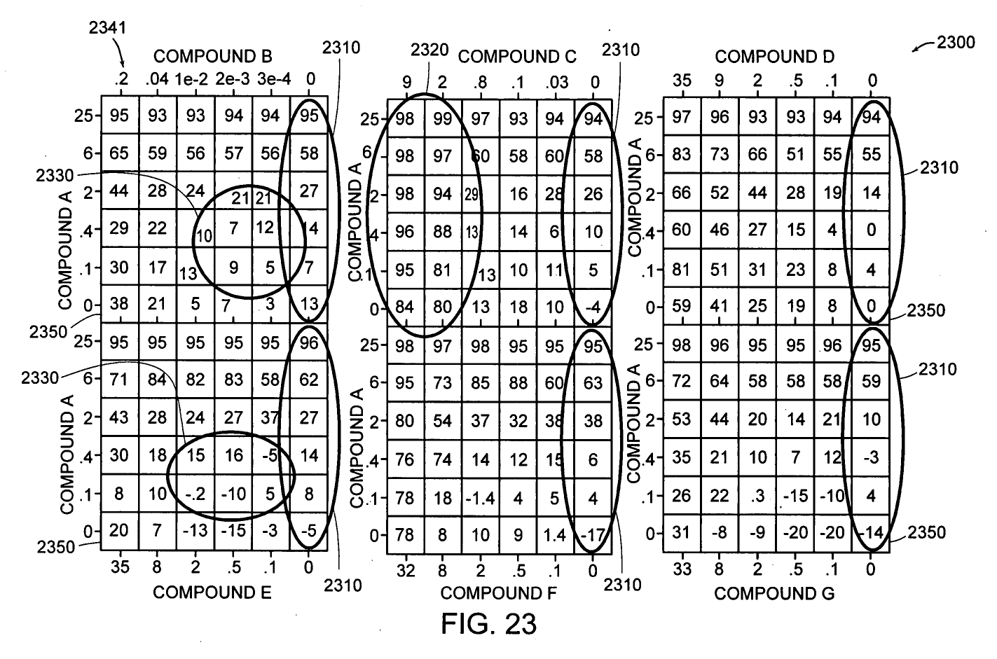

FIG. 23 presents depicts values of inhibition associated with locations of an assay array in the form of six 6×6 subarrays. Each row of each subarray contains a particular concentration of entity A. Each column of a particular subarray contains a particular concentration of another entity. Each subarray utilizes a different entity which is combined with entity A to create the combined composition in the subarray. For example, one subarray 2341 utilizes varying concentrations of entity B in each column. Another subarray 2342 utilizes varying concentrations of entity C in each column.

Examining the inhibition values of the six subarrays shows particular inefficiencies and redundancies in the data collected regarding inhibition values. For example, each subarray contains a column 2310 that represents the single agent values of inhibition that are associated with entity A (i.e., these columns represent locations where the concentration of the column entity is zero). Thus the single agent data is repeated six times. Furthermore, rows of each subarray 2350 are associated with single agent inhibition values of the entities that are combined with entity A (though in those particular rows the concentration of entity A is zero). Thus in a complete experiment, these row values 2350 would be repeated each time the designated entity is combined with another constituent composition. As well, some locations of the subarrays 2330 show values of inhibition that are so low that a synergistic effect is unlikely to be present. Other locations of the subarrays 2320 show values of inhibition that are so high that a synergistic effect is unlikely to be present. The effect of repetition of single agent values and of activity determination in locations where the concentration of the agents is either too high or too low shows the potential inefficiencies of this particular assay array arrangement.

a. Concentration Selection in Constituent Arrays Based Upon Solo Constituent Composition Activity

Experience in testing has led to the finding that when constituent compositions are combined, the vast majority of synergetic results (i.e., instances where the combined combination has an effect above that expected for the effect of the single agents acting independently) in the combined composition are located in the region where each constituent composition is in its transition zone, i.e., the concentration range where the activity of a given constituent composition, acting in solo, changes most rapidly as a function of concentration of one or more entities of the constituent composition. For example, when activity of a constituent composition is gauged in terms of inhibition, the transition zone may cover a range of concentrations corresponding to approximately 20% to 80% of the maximum inhibition exhibited by constituent composition acting alone at any concentration.

Thus, in order to increase the utility of experimental data gathered concerning the activity of a combined composition, embodiments of the invention may utilize one or more constituent compositions of a combined composition within the assay array at a concentration corresponding to a designated activity level of the constituent composition acting alone. This is in contrast to embodiments of the invention that may utilize concentrations of constituent compositions based upon some dilution from a designated maximum value without regard to the activity of the constituent composition.

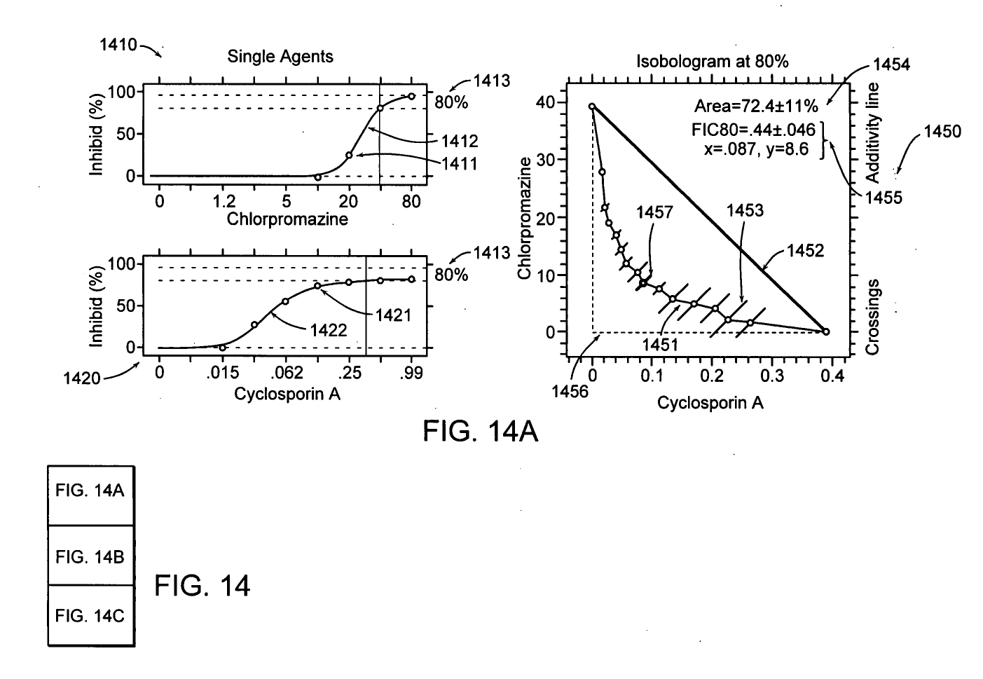

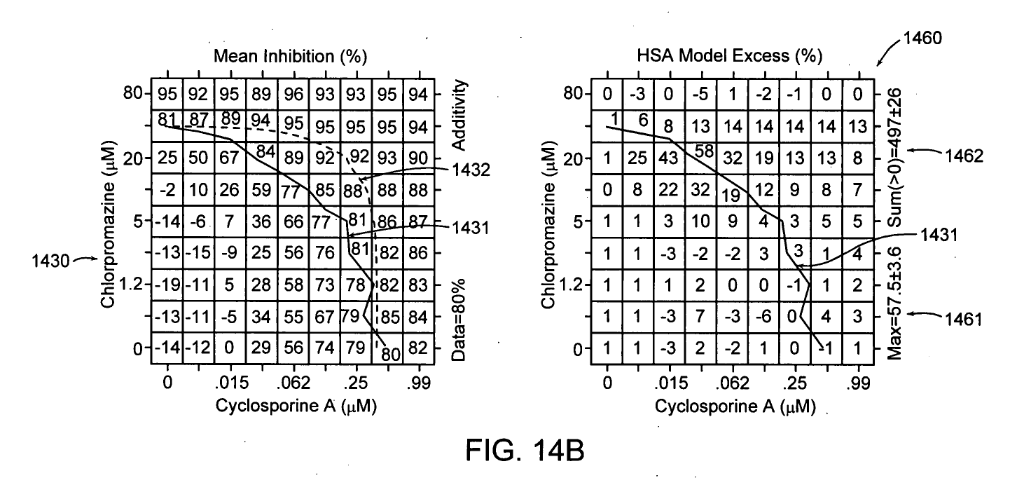

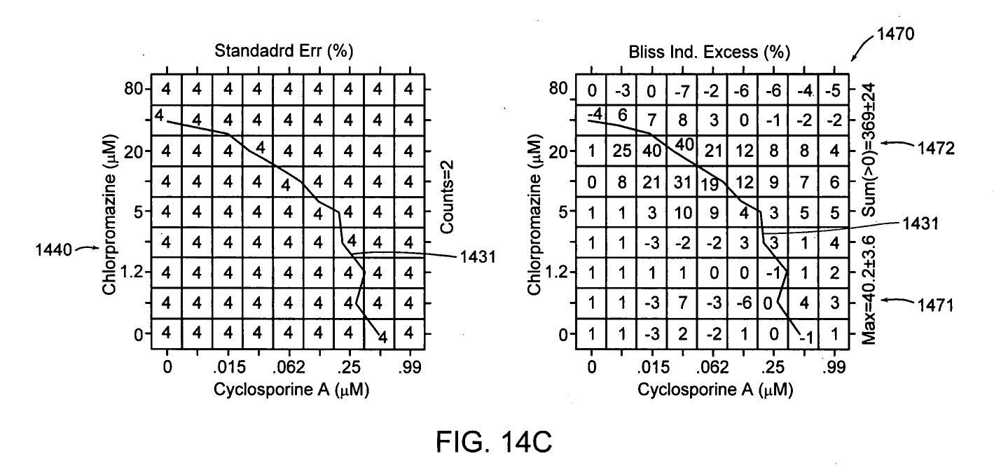

Data concerning constituent composition activity acting alone may be gathered from any source. Such data may be already known in the literature or from past experiments. In some embodiments of the invention, data concerning the individual constituent composition activity may be gathered through an evaluation in an assay experiment before the combined compositions are evaluated. Data gathered may be plotted in terms of activity versus concentration, a specific example shown in graphs 1410 and 1420 in FIG. 14, to obtain the necessary concentrations for designated values of activity.

In embodiments of the invention that utilize values of inhibition as a measure of activity, transition zone inhibitions correspond to values typically occurring in the approximate range of 20% to 80% of the maximum inhibition exhibited by the constituent composition at any concentration. Thus, based upon solo constituent composition inhibition values, the concentrations of an active agent in a constituent array may be chosen such that the concentrations correspond to designated values of inhibition in the approximate range of 20% to 80% of the maximum possible inhibition. For example, in a 6×6 assay array in which two constituent compositions are combined, the six concentrations of each constituent composition may correspond to concentrations where the value of inhibition may correspond approximately to 0%, 20%, 40%, 60%, 80%, and 100% of the maximum inhibition for each of the individual constituent compositions. Of course, other fractions of the maximum value of inhibition may also be used to determine the relevant concentrations in other embodiments of the invention.

In a preferred embodiment, some concentrations of a constituent composition utilized in an assay array are designated as the product of a multiplicative factor and a concentration corresponding to a given activity level. For example, for a 6×6 assay array in which activity is gauged by a value of inhibition, a concentration corresponding to approximately 80% of the maximum inhibition for the activity of a particular constituent composition may serve as a baseline concentration. A two-fold, four-fold, and eight-fold dilution from the baseline concentration may be utilized to identify three other concentrations to be utilized for evaluation, i.e., a factor of two is utilized for the multiplicative factor. A factor of two often suffices to give good results. The final two concentrations are, typically, zero concentration and a concentration resulting in approximately 100% of the maximum inhibition. In some instances, for example when the concentration versus inhibition curve of a constituent composition exhibits a sigmoidal-like shape, the concentration associated with a slightly lower than maximum inhibition (e.g., 99% of maximum inhibition) is utilized instead of the maximum inhibition concentration in some embodiments of the invention.

In the aforementioned example, the baseline concentration serves to mark the approximate edge of the transition zone. The multiplicative factor provides a simplified methodology for determining additional concentrations to examine throughout the transition zone. Of course, other ways of choosing a baseline concentration, or determining the multiplicative factor, may be utilized. In one example utilizing a 6×6 assay array, the chosen concentrations of the constituent compositions are zero concentration and concentrations corresponding to 20%, 80%, and 100% of maximum inhibition for the constituent composition. The remaining two concentrations are evenly distributed between the 20% and 80% of maximum inhibition concentrations. Using a multiplicative factor of

$$\sqrt[3]{\frac{conc . assoc . with80 \% of max . inhibition}{conc . assoc . with20 \% of max . inhibition}}$$

one concentration is the product of the multiplicative factor and the concentration corresponding to 20% of maximum inhibition. The remaining concentration is the product of the square of the multiplicative factor and the concentration corresponding to 20% of maximum inhibition. In another example, the concentration associated with the bottom edge of a transition zone is determined; multiplying the identified concentration with a multiplicative factor greater than one may generate the other concentrations. As well, other ways of utilizing a baseline concentration to determine other concentrations for the constituent composition may be utilized (e.g., a geometric factor) depending upon the nature of the constituent composition.

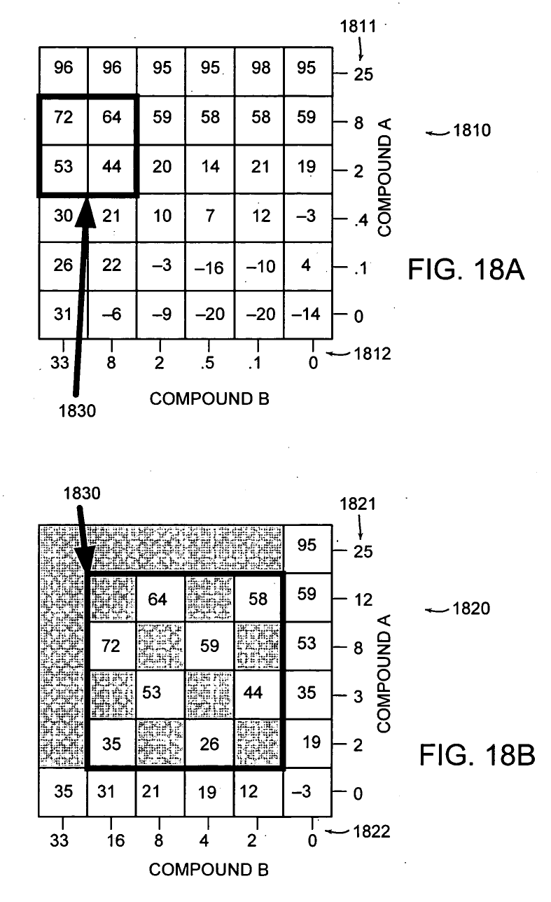

FIGS. 18A and 18B depict some of the advantages of selecting particular concentrations for the constituent composition as discussed earlier. In FIG. 18A, the array 1810 depicts inhibition values of combining composition A with composition B. The rows of the array 1810 represent locations with constant concentration of composition A, each row being a different concentration of composition A as designated on the Y-axis 1811. Similarly, the columns of the array 1810 represent locations with constant concentration of composition B, each column being a different concentration of composition B as designated on the X-axis 1812. As designated by the 4 locations marked 1830 in the array 1810, only 4 of the 36 locations provide data regarding the possible synergetic effects of combining compositions A and B.

In contrast, FIG. 18B depicts an array 1820 in which the concentrations of composition A and B are chosen by identifying a baseline concentration for each composition and diluting by a multiplicative factor. In particular, the concentrations of composition A, as marked on the Y-axis 1821, correspond to percentages of the maximum inhibition of substantially 0%, 100% and approximately 80%. The remaining three concentrations correspond to approximate multiples of two-fold dilutions from the approximately 80% of maximum inhibition concentration. Similarly, the concentrations of composition B, as marked on the X-axis 1822, is similarly chosen. The expanded number of locations 1830 in the array 1820 represent a substantial increase in the amount of data that may be used to identify a combination effect.

Concentration selection, as discussed above, may also be implemented to detect other combination effects beyond a synergistic effect. For example, enhanced antagonism effects may be more prevalent for combinations of constituent compositions where the active agents are present in a higher range of their constituent composition effect concentrations. Thus, in terms of inhibition, a combination surface may be probed in more detail at higher concentrations of the individual candidate compositions than is typically utilized in searching for synergistic effects. Similarly, a lower concentration range associated with small values of the maximum inhibition of a constituent composition may also be probed when appropriate.

Though embodiments of the invention related to concentration selection as discussed herein refer to specific values of activity, such as percentages of maximum inhibition, it should be clear to those skilled in the art that concentrations related to precise values of activity are not required to practice such embodiments. Indeed, concentrations and values of activity need only be within an approximate range for use in such embodiments; since the embodiments of the invention are directed toward probing the range of the transition zone of a constituent composition, and not specific points in the range, precise values of the activity are not necessary to practice such embodiments.

Embodiments of the invention that utilize the concentration selection procedures discussed herein include any manner of preparation of constituent arrays that eventually are combined to form assay arrays. Thus, for example, concentration selection may be used in conjunction with embodiments of the invention that utilize origin and derivative sets, dilution arrays, or constituent arrays that are configured on multiple physical objects. In instances when intermediate arrays, such as a dilution array or portions of an assay array, are used which result in a dilution for each separate array produced before an array of combined compositions is evaluated for activity, embodiments of the invention are configured such that concentrations of constituent compositions corresponding to a designated activity of the constituent composition are the final concentrations in the evaluated locations of the assay array.

In a preferred embodiment of the invention, concentration selection is utilized in conjunction with the virtual sparse array techniques discussed below to provide enhanced efficiency in evaluating combined compositions.

b. Assay Array Configurations Corresponding to a Virtual Sparse Array

As exemplified in FIG. 23, particular assay array configurations (e.g., assay array 2300) may duplicate data unnecessarily, leading to inefficiencies in evaluating the activity in an assay array. Furthermore, in particular situations not all of an assay array need be evaluated to obtain information regarding a combination effect between combined compositions. For example in FIG. 18B, the use of concentration selection enlarges the number of locations 1830 of the assay array 1820 which may be used to detect combination effects. However, not all the assay array 1820 need be evaluated to provide a measure of a combination effect in the assay array. Indeed, not even all the locations associated with detection of a combination effect 1830 need be evaluated. As depicted by the filled numerical locations of the combination effect region 1830, evenly distributed spacing of evaluated locations may provide sufficient data to detect combination effects.

Thus, some embodiments of the invention discussed herein configure constituent arrays to create assay arrays that have combinations in locations that correspond to the filled locations of the assay array 1820 shown in FIG. 18B. Since the actual assay array may be densely packed (i.e., no skipped locations may actually exist in the actual assay array), we say that the actual assay array locations correspond to the locations of a “virtual sparse assay array” (e.g., the form of the array 1820 in FIG. 18B). In such instances, assay arrays may be created that do not combine every concentration of a constituent composition on a constituent array with every other concentration of a constituent composition on a different constituent array. That is, a given concentration of a constituent composition in an assay array is not combined with every concentration of any other constituent composition utilized in the assay array.

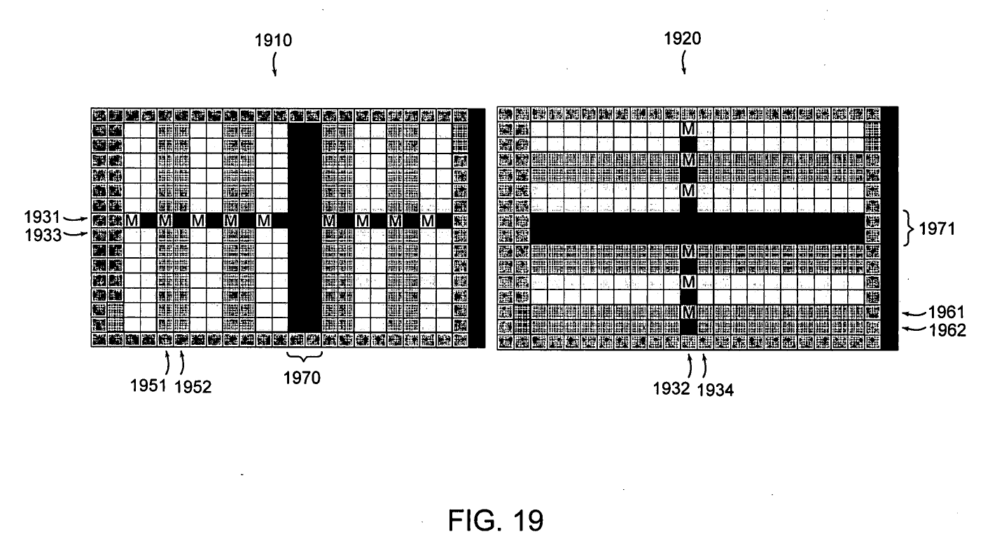

FIG. 19 depicts the configuration of two constituent arrays 1910, 1920 that may be utilized in a particular embodiment of the invention to create an assay array that also corresponds to a virtual sparse array. In the column constituent array 1910, the two columns adjacent to the ends of the array and the rows adjacent to the edge are not utilized. The locations of row 1931 of the constituent array 1910 are utilized as control locations. Sets of adjacent pairs of columns, for example the columns 1951, 1952 of FIG. 19, contain the same constituent composition with the exception of edge locations and locations corresponding with the intersection of the control row 1931. Each location in a column has the same concentration of constituent composition. Each column of the pair, however, has a different concentration of the constituent composition. For example, column 1951 contains a concentration of constituent composition in each location which is diluted to ⅕ the maximum concentration of the constituent composition used. In the location designated by “M”, however, the concentration of the constituent composition is the maximum concentration of the constituent composition utilized in the columns 1951, 1952. For column 1952, the concentration of constituent composition is ⅗ the maximum concentration of the constituent composition. In the intersection location with the control row, however, the location contains a control composition.

Every other pair of columns in the constituent array 1910 is similarly arranged, each pair of columns typically associated with a different constituent composition. The left hand column of each pair contains ⅕ the maximum concentration of the constituent composition with the location intersecting the control row 1931 containing the maximum concentration of constituent composition. The right hand column of each pair contains ⅗ the maximum concentration of the constituent composition with the location intersecting the control row 1931 containing a control composition. Columns 1970, however, are unfilled.

The row constituent array 1920 is configured in a similar fashion to the column constituent array 1910, albeit in a column format. Again, the two columns adjacent to the ends of the array and the rows adjacent to the edge are not utilized. The locations of column 1932 of the constituent array 1920 are utilized as control locations. Sets of adjacent pairs of rows contain the same constituent composition with the exception of edge locations and locations corresponding with the intersection of the control column 1932. Each location in a row has the same concentration of constituent composition. Each row of the pair, however, has a different concentration of the constituent composition. For example, row 1961 contains a concentration of constituent composition in each location which is diluted to ⅘ the maximum concentration of the constituent composition used. In the location designated by “M”, however, the concentration of the constituent composition is the maximum concentration of the constituent composition used in the rows 1961, 1962. For row 1962, the concentration of constituent composition is ⅖ the maximum concentration of the constituent composition. In the intersection location with the control column 1932, however, the location contains a control composition.

All other pairs of rows in the constituent array 1920 are similarly arranged, each pair of rows typically associated with a different constituent composition. The upper row of each pair contains ⅘ the maximum concentration of the constituent composition with the location intersecting the control column 1932 containing the maximum concentration of constituent composition. The lower row of each pair contains ⅖ the maximum concentration of the constituent composition with the location intersecting the control column 1932 containing a control composition. Rows 1971, however, are unfilled.

Corresponding locations of the constituent arrays 1910, 1920 are combined in a corresponding location of an assay array 2010, as depicted in FIG. 20. Rows 2018 are the result of combining the corresponding locations of rows 1931, 1933 with rows 1971. Since the rows 1971 are unfilled, rows 2018 substantially match the contents of rows 1931, 1933. For example, the locations 2011 correspond to a constituent composition in rows 1931, 1933 having the maximum concentration, ⅕ the maximum concentration, ⅗ the maximum concentration, and a control composition. Similar groups of four locations along rows 2018 provide the same groupings of compositions, though for a particular constituent composition associated with a particular pair of columns.

In a similar fashion, columns 2016 are the result of combining the corresponding locations of columns 1932, 1934 with columns 1970. Continuing the example discussed in FIG. 19, the locations 2013 of FIG. 20 correspond to a constituent composition in columns 1932, 1934 having the maximum concentration, ⅖ the maximum concentration, ⅘ the maximum concentration, and a control composition. Similar groups of four locations along columns 2016 provide the same groupings of compositions, though for a particular constituent composition associated with a particular pair of rows.

Rows 2018 and columns 2016 thus provide locations corresponding to pure constituent composition activity data, and data related to controls. The latter data may also be used for assay controls and plate effect correction as discussed elsewhere, while the former data may be used for both composition controls and as a source of single agent data for performing analysis regarding combination effects such as a global c-value test.

The intersection of any pair of columns, with correspondence to columns having the same constituent composition in array 1910, and any pair of rows, with correspondence to rows having the same constituent composition in array 1920, in the assay array 2010 provides 4 locations containing values of combined compositions. For example, the locations 2012 of the assay array 2010 correspond to the four possible pairwise combinations of compositions between the constituent composition in locations 2011 corresponding to concentrations that are ⅕ and ⅗ of the maximum concentration, and the constituent composition in locations 2013 corresponding to concentrations that are ⅖ and ⅘ of the maximum concentration.

The data in locations 2011, 2012, 2013 of assay array 2010 provide a portion of the locations that are typically present in a more complete assay array format. For example, virtual assay array 2020 represents an assay array that presents locations having every possible pairwise combination of only two of the constituent compositions in assay array 2010, each constituent composition having a concentration of zero, ⅕, ⅖, ⅗, ⅘, and 5/5 of a maximum concentration. If the two constituent compositions are the compositions utilized in locations 2011, 2012, 2013, the filled squares of the virtual assay array 2020 are the data known from the locations. Thus the locations 2011, 2012, 2013 act as locations of a “virtual sparse array” as shown by assay array 2020.

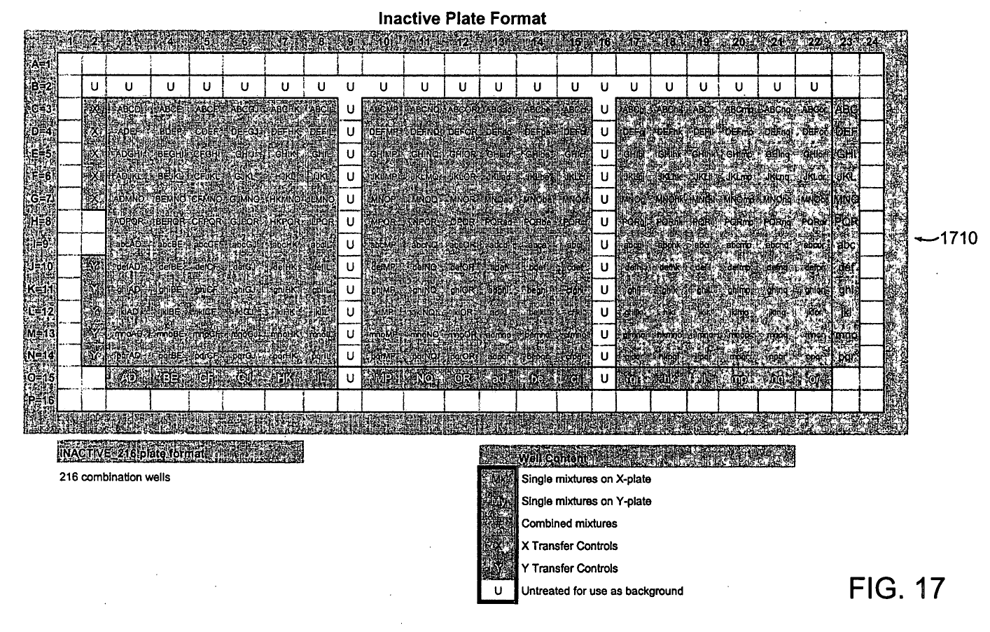

Some advantages of using a format as presented in assay array 2010 are evident in comparing the array with a more complete virtual assay array 2020 for only two constituent compositions. First, a substantial fraction of the data concerning combined compositions in the virtual assay array 2020 is covered by the choice of the concentrations of the constituent compositions. Second, assay array 2010 covers a much larger number of pairwise combinations of constituent compositions. Assay array 2010 provides data on 54 pairs of constituent compositions. An equivalent number of locations distributed for the more complete 6×6 format would not even allow the complete testing of 8 pairs of constituent compositions. Third, the configuration of the control compositions and pure constituent composition data reduce the duplication inherent in the more complete assay arrays, as depicted by locations 1710 in FIG. 17.

In another embodiment of the invention related to assay arrays corresponding to a virtual sparse arrays, each of arrays 1910, 1920 may be considered only part of a larger constituent array. As well, the resulting combined array 2010 may also be a portion of a larger assay array. A new column array may be formulated identically to column array 1910 except that the concentrations of constituent composition are at ⅖ or ⅘ of the maximum concentration in each column, as opposed to ⅕ or ⅗ of the maximum concentration. The new column array and array 1910 constitute the total column constituent array. Analogously, a new row array is formulated identically to row array 1920 except that the concentrations of constituent composition are at ⅕ or ⅗ of the maximum concentration in each row, as opposed to ⅖ or ⅘ of the maximum concentration. The combination of the new row array and array 1920 is the total row constituent array.

The combining of corresponding locations of the new row array and new column array results in a new combination array which has similar structure to combination array 2010. For example, the locations in the new combination array, corresponding to locations 2011, 2012, 2013 of array 2010, map onto the filled spaces of virtual array 2030. The locations with constituent compositions do not overlap the locations that are filled in the virtual array 2020. The union of the filled locations from the new combination array and the corresponding locations of the combination array 2010 form the corresponding locations of the total assay array. Furthermore, virtual array 2040 depicts the information contained by combining the corresponding locations 2011, 2012, 2013 of the two combination arrays. Thus as depicted in the array 2040, the total assay array provides all the pure constituent composition data in the more complete virtual array for a given pair of constituent compositions, and an offset, alternating pattern of filled locations for the possible pairwise combination of the constituent compositions at the various concentrations of the constituent arrays.

The ability of utilizing a sparse matrix format to detect synergetic combinations was tested using existing combination data. A simulation was performed using data on 92 compounds that were pairwise combined at different concentrations. The data was manually analyzed to determine combinations of the compounds at various concentrations that exhibited a synergistic interaction. An automated method of identifying synergistic combinations, as discussed earlier, is applied to the data in two simulations.

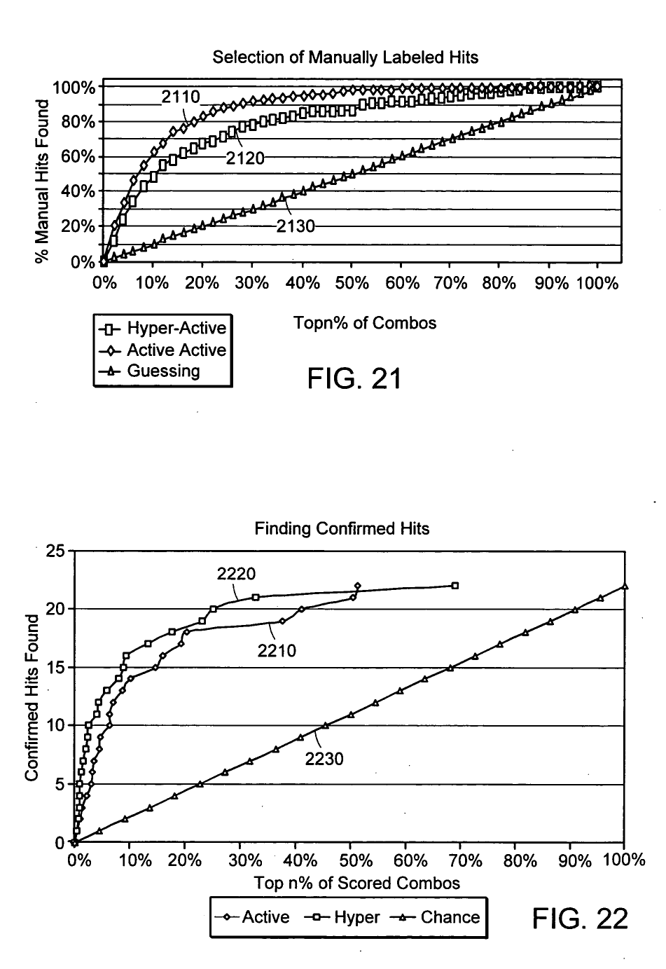

First, the automated method was applied to the data in which the data was complete enough to fill every location of an array of the form 2020, 2030, 2040 for every possible combination of constituent compositions, i.e., every possible pairwise combination of constituent composition for every concentration was examined by the method. Graph 2110 of FIG. 21 presents the results of the automated method as applied to every possible combination. The graph presents the percentage of synergistic hits that were located by the method as a function of the percentage of the highest scores examined by the method.

The automated method was applied a second time to the data. In this instance, however, only pairwise combinations that correspond to the filled locations of a virtual array as presented in array 2040 were analyzed by the method, i.e., some combinations of constituent compositions at particular concentrations corresponding to the empty squares of array 2040 were not analyzed by the method. Graph 2120 of FIG. 21 presents the results of the second simulation. Graph 2130 represents the possibility of locating a synergistic combination based upon random chance guessing.

For a given percentage of the top combinations viewed, the second simulation, which represents a sparse array configuration, finds nearly as many of the manual hits as the more complete search of all the data in the first simulation. However, given the far fewer number of locations that need to be evaluated in an assay array for a sparse configuration, benefits in efficiency may be obtained.

In a related preferred embodiment of the invention, the sparse array configuration previously described is combined with the concentration selection techniques to provide enhanced efficiency in identifying combination effects in combined compositions. In particular, the concentrations utilized in a row array 1920 or a column array 1910 may be configured such that upon transfer of corresponding contents to an assay array the concentration selection criteria of choosing concentrations in the transition zone of activity of the individual constituent compositions is met. For example, the locations designated “M” in the arrays 1910, 1920 may correspond to a concentration of constituent composition necessary to achieve 99% of the maximum inhibition that the constituent composition is capable of achieving. Locations that were formerly designated to contain ⅘ of the maximum concentration of a constituent composition are designated to contain a concentration that provides 80% of the maximum inhibition of the constituent composition to the assay array upon transfer. The locations formerly holding ⅗, ⅖, and ⅕ of the maximum concentration are now designated to hold concentrations corresponding to 60%, 40% and 20% of the maximum inhibition of the constituent composition, respectively, upon appropriate transfer to the assay array. Of course, other designations for concentration selection (e.g., using a factored dilution from a particular activity level) may also be utilized in place of specific percentages of maximum inhibition.

Combining the row and column arrays results in combination arrays that have implemented concentration selection. The effectiveness of combining sparse array techniques with concentration selection is evaluated in another test. The 92 combinations of constituent compositions at varying concentrations were experimentally evaluated for combination effects using sparse array techniques and concentration selection. The efficiency of the full evaluation technique described in the last test (i.e., pairwise combining every concentration of every constituent composition without utilizing the concentration selection techniques) was compared with the efficiency of using a sparse array with concentration selection. A total of 22 synergistic combinations were present in all possible combinations based upon an independent experimental evaluation of possible combinations.

The ability of each evaluation technique to detect all 22 synergistic combinations is shown in FIG. 22. Graph 2210 represents the number of the synergistic combinations that are located for a given percentage of the highest scored examined in the full evaluation method. Graph 2220 presents the results obtained using data from a sparse array with concentration selection. Graph 2230 represents the probability of obtaining the hits on the basis of random choice. FIG. 22 shows that use of a sparse array with concentration selection is generally more efficient at locating the synergistic combinations than the full evaluation method.

Variations of arrays that correspond to a virtual sparse array will be apparent to those skilled in the art. The scope of the invention is in no way limited to the specific embodiments discussed earlier. For example, different sizes of arrays (beyond the 6×6 arrays described earlier), and different configurations of locations of combined compositions may be utilized. As well, various selections of concentration ranges for the constituent arrays, and the ordering of such concentrations on each portion, or the entirety, of a constituent array are within the scope of the invention. In another example, “M” need not correspond with a “maximum” concentration but rather some reference based concentration of the constituent composition.

Other embodiments of the invention may configure the control rows and control columns of arrays around the edges of the arrays, or in discrete sections in different locations of an array. In another alternative embodiment, constituent arrays need not necessarily be ordered as one or more row arrays or column arrays, but may take any form convenient to a user. Row arrays or column arrays that are similarly configured, except for the concentrations of the constituent composition, may be embodied on separate physical entities or all on one physical entity.

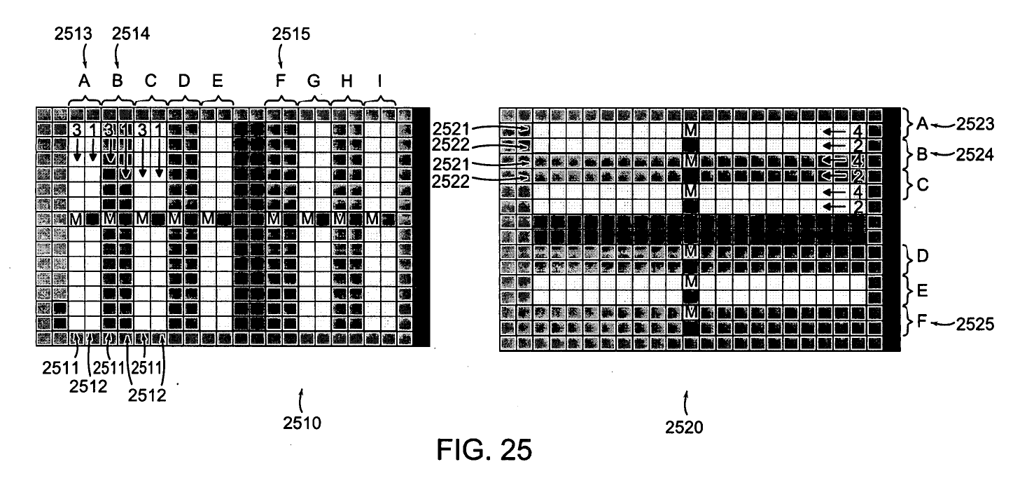

As one example of some of the variations described above, FIG. 25 depicts a column constituent array 2510 and a row constituent array 2520 utilized in a particular embodiment of the invention. Each constituent array contains a series of control locations laid out similarly to the arrays 1910, 1920 depicted in FIG. 19. Also as depicted in FIG. 19, locations designated with an ‘M’ correspond to locations having a maximum concentration of a particular constituent composition.

Column constituent array 2510 contains a series of pairs of columns 2513, 2514, 2515. Each pair of columns contains a constituent composition as designated A through I along the top of the constituent array 2510. For each pair of columns corresponding to a particular constituent composition, the left hand columns 2511 correspond to locations having a concentration of particular constituent composition approximately equal to ⅗ of the maximum concentration of the particular constituent composition in the column array 2510. The right hand columns 2512 correspond to locations having a concentration of particular constituent composition approximately equal to ⅕ of the maximum concentration of the particular constituent composition in the column array 2510.

Row constituent array 2520 contains a series of pairs of rows 2523, 2524, 2525. Each pair of rows contains a constituent composition as designated A through F along the right hand side of the constituent array 2520. For each pair of rows corresponding to a particular constituent composition, the top rows 2521 correspond to locations having a concentration of particular constituent composition approximately equal to ⅘ of the maximum concentration of the particular constituent composition in the row array 2520. The bottom rows 2522 correspond to locations having a concentration of particular constituent composition approximately equal to ⅖ of the maximum concentration of the particular constituent composition in the row array 2520.

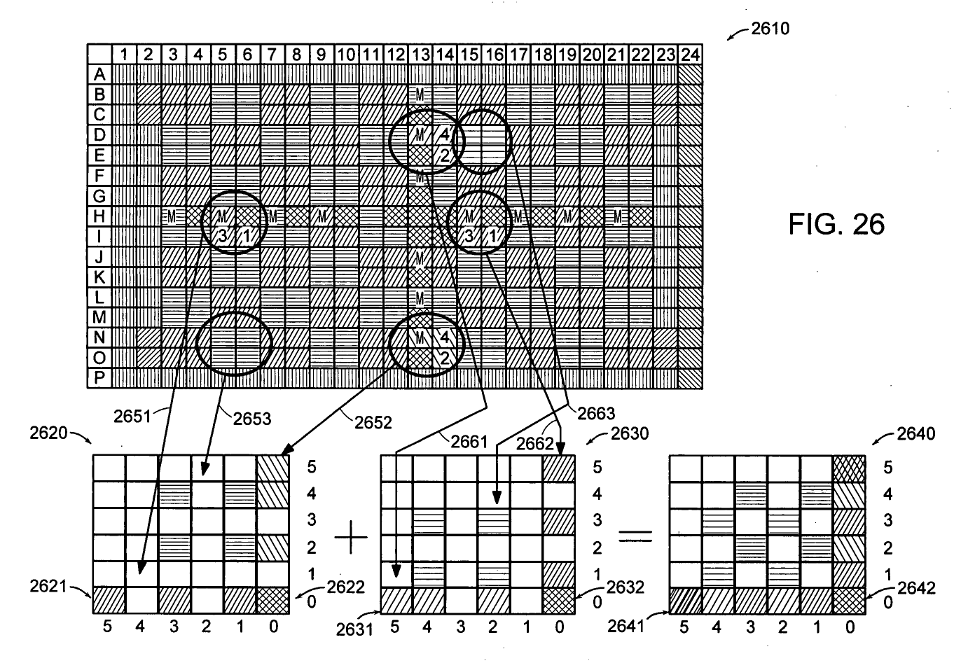

FIG. 26 depicts an assay array 2610 resulting from combining the corresponding locations of the column constituent array 2510 and the row constituent array 2520. The 4 locations 2653 of the assay array 2610 are the result of combining composition B from the columns 2514 of the column constituent array 2510 with composition F from the rows 2525 of row constituent array 2520. Note that the pure constituent compositions in their corresponding concentrations are present in the bottom 2 locations of 2651 (composition B) and the right hand locations of 2652 (composition F).

Virtual combination array 2620 depicts an array with locations corresponding to all possible pairwise combinations of compositions B and F at every concentration utilized in the constituent arrays 2510, 2520, as well as locations corresponding to the pure constituent compositions at the various concentrations. The pure composition F locations 2652 map to the filled locations of the right hand column 2622 of the virtual array 2620. The pure composition B locations 2651 map to the filled locations of the bottom row 2621 of the virtual array 2620. The combined compositions of B and F of locations 2653 map to the inner 4 locations of the virtual array 2620.

The use of compositions B and F in both the column constituent array 2510 and the row constituent array 2520 at different concentrations leads to assay array 2610 resulting in further locations that can fill further locations of the corresponding virtual array of combinations of compositions B and F. The 4 locations 2662 of the assay array 2610 are the result of combining composition F from the columns 2515 of the column constituent array 2510 with composition B from the rows 2524 of row constituent array 2520. Again, the pure constituent compositions in their corresponding concentrations are present in the bottom 2 locations of 2662 (composition F) and the right hand locations of 2661 (composition B).

Virtual array 2630 contains filled locations corresponding to locations 2661, 2662, 2663 of the assay array 2610. The pure constituent composition F locations 2662 map to the filled right hand column locations of the virtual array 2630, while pure constituent composition B locations 2661 map to the filled bottom row locations of the array 2630. The combination locations 2663 map to the remaining filled locations of the virtual array 2630.

Note that layout of the constituent arrays 2510, 2520 and the assay array 2610 are configured such that no overlap of constituent composition data exists between the virtual arrays 2620, 2630. Thus, the combined virtual array 2640, which assembles all the corresponding filled locations in the arrays 2620, 2630, contains all the pure constituent B locations 2641 at each concentration, all the pure constituent F locations 2642 at each concentration, and mixtures of combinations of the various concentrations of compositions B and F. Thus this embodiment of the invention is capable of providing a virtual sparse assay array that contains pairwise combinations of compositions A-F, as well as some other combination data.

The number of rows or columns used to represent a particular constituent composition on a row array or column array may be varied to alter the size and density of the assay array. For example, in embodiments of the invention previously described herein, pairs of row and pairs of columns were utilized. However, other embodiments of the invention may use other numbers (e.g., grouping 4 rows or columns together for each constituent composition in a row or column array).

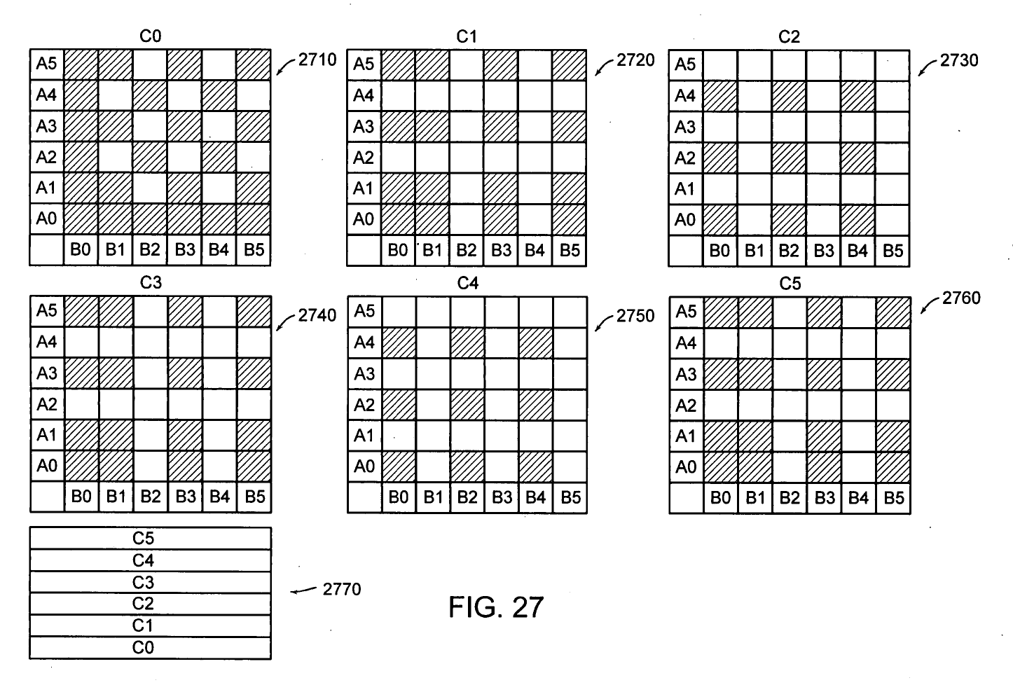

The sparse assay array configuration may also be utilized in a three dimensional format in which combinations of 3 constituent compositions are combined. One such embodiment of the invention in depicted in FIG. 27, which shows various aspects of a virtual sparse array configured as a three-dimensional cube of combinations of entities A, B, and C. Each of arrays 2710, 2720, 2730, 2740, 2750, 2760 correspond to virtual two dimensional arrays of combinations of varying concentrations of entity A and B, with a particular concentration of entity C in a plurality of the locations. The two dimensional arrays 2710, 2720, 2730, 2740, 2750, 2760 are stacked as a three dimensional array 2770. The three-dimensional virtual array 2770 is sparse not only in the two dimensions of concentrations of entities A and B, but also in the stacking dimension since the filled locations of each two dimensional slice do not coincide. The methods previously described herein for constructing constituent arrays and assay arrays may be applied to construct a resulting three-dimensional virtual array.

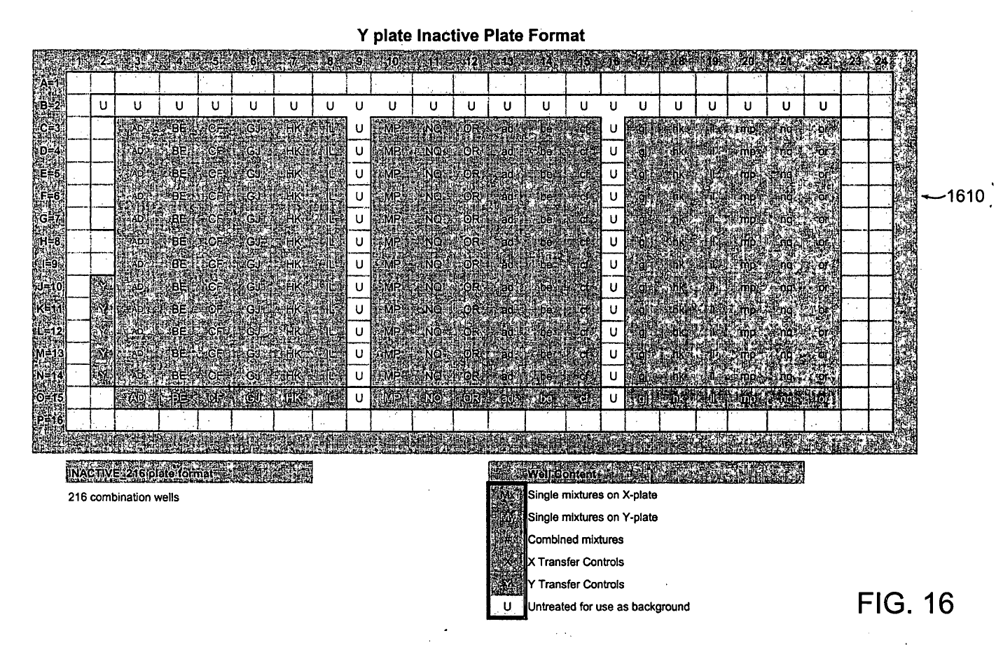

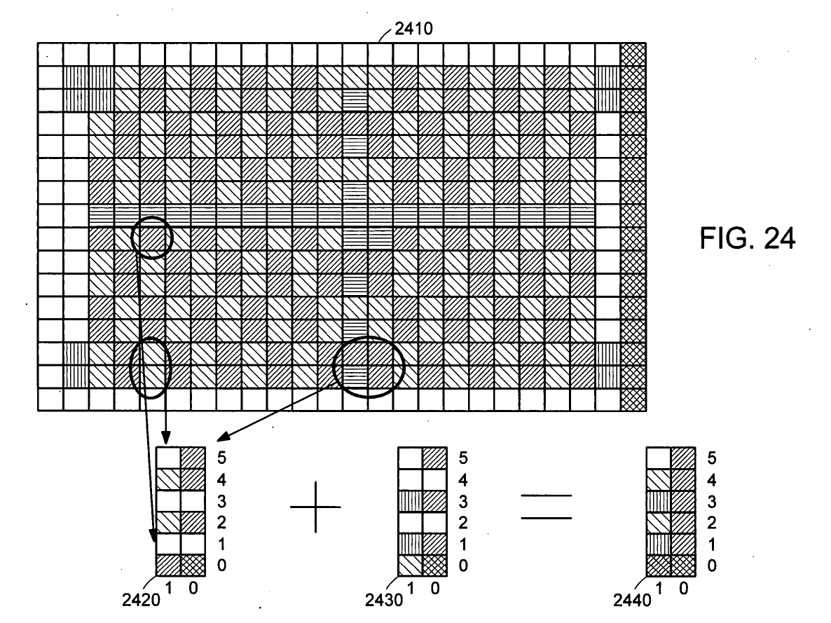

In another embodiment of the invention, a constituent array may be configured to prepare a sparse array, while another constituent array may be configured in another format. As shown in FIG. 24, combination array 2410 is the result of combining a row array in the format of array 1920 with a column array in which each column has a high concentration of several entities (e.g., the format shown in the array 1610 of FIG. 16), all locations in a column having an identical composition (with the exception of the edges and control positions). Virtual array 2420 shows the portion of a complete array that corresponds with the appropriate locations of the combination array 2410. Another combination array formed from a column array that is formatted to be sparse with a row array similar to array 1510 (with appropriately placed control locations). The new combination array provides data on other locations of the virtual array as depicted by array 2430, the total combined data being presented on array 2440.

Though the embodiments described above refer to detecting phenomena corresponding to inhibition, those skilled in the art of assay testing will readily recognize that the techniques discussed are applicable in other contexts as well.

### EXAMPLES

The following examples are provided to illustrate some embodiments of the invention. The examples are not intended to limit the scope of any particular embodiment utilized.

### Example 1

### Assay for Proinflammatory Cytokine-Suppressing Activity

In this example, we assay a mixture of chlorpromazine and cyclosporine A at various dilutions for the suppression of phorbol 12-myristate 13 acetate/Ionomycin stimulated IL-2 and TNF-α secretion from human white blood cells using the ELISA method, as described below. In accordance with the definition of terms provided earlier in this description, each compound is an “entity”, and each mixture of the two entities is a “candidate composition” (for purposes of illustration in examples 1 and 2, the first use of a defined term appears in quotation marks). When the components of the assay, which are collectively known as an “evaluative composition”, are added to each mixture, we have a “combined composition” (note, however, that “combined composition” is broad enough to include a candidate composition by itself).

“Arrays” are embodied as plates with wells in this example. A set of “origin” locations of a “constituent array” containing chlorpromazine is prepared as a Y array on a plate, wherein chlorpromazine is successively diluted in the direction of the columns of the plate, each row having the same concentration of chlorpromazine. As well, a set of origin locations of a constituent array containing cyclosporine A is prepared as an X array on a plate, wherein cyclosporine A is successively diluted in the direction of the rows of the plate, each column having the same concentration of cyclosporine A. For each of the X and Y arrays, a portion of the contents of each well is transferred to the corresponding wells of another plate, with diluent; the corresponding wells representing a set of corresponding “derivative” locations for the constituent array. A portion of the contents of the wells of each plate holding a derivative set is transferred to corresponding locations of a plate, with diluent, to form an “assay array”. Each well of the assay array is evaluated for the activity of the candidate composition, i.e. the ability of the particular mixture of chlorpromazine and cyclosporine A to suppress phorbol 12-myristate 13 acetate/Ionomycin stimulated IL-2 and TNF-α secretion from human white blood cells using the ELISA method.

Preparation of Compounds

The stock solution containing chlorpromazine was made at a concentration of 10 mg/ml in DMSO, and the stock solution containing cyclosporine A was made at a concentration of 1.2 mg/ml in DMSO. Plates with wells arranged in a 9×9 matrix, corresponding to the set of origin locations of a constituent array 830, were prepared following the configuration shown in FIG. 8 and stored at −20° C. until ready for use. Chlorpromazine was successively diluted in columns of its plate. Cyclosporine A was successively diluted in rows of its plate.

As shown in FIG. 5, the single agent plates containing the derivative sets corresponding to each origin set 511 and 521 were generated by transferring 1 μL of stock solution from the specific plate containing a particular origin set 510, 520 to separate plates 511 and 521 containing 100 μL of media (RPMI; Gibco BRL, #11875-085), 10% fetal bovine serum (Gibco BRL, #25140-097), 2% penicillin/streptomycin (Gibco BRL, #15140-122)) using the Packard Mini-Trak liquid handler. The plates containing the derivative sets 511 and 521 were then combined, a 10 μL aliquot transferred from each plate 511, 521 to the final assay plate 531 (polystyrene 384-well plate (NalgeNunc)), which was pre-filled with 30 μL/well RPMI media containing 33 ng/mL phorbol 12-myristate 13-acetate (Sigma, P-1585) and 2.475 ng/mL ionomycin (Sigma, I-0634).

IL-2 Secretion Assay

The effects of test compound combinations on IL-2 secretion were assayed in white blood cells from human buffy coat stimulated with phorbol 12-myistate 13-acetate, as follows. Human white blood cells from buffy coat were diluted 1:50 in media (RPMI; Gibco BRL, #11875-085), 10% fetal bovine serum (Gibco BRL, #25140-097), 2% penicillin/streptomycin (Gibco BRL, #15140-122)) and 50 μL of the diluted white blood cells was placed in each well of the final assay plate created in the above section. After 16-18 hours of incubation at 37° C. in a humidified incubator, the plate was centrifuged and the supernatant was transferred to a white opaque 384-well plate (NalgeNunc, MAXISORB) coated with an anti-IL-2 antibody (PharMingen, #555051). After a two-hour incubation, the plate was washed (Tecan Powerwasher 384, Tecan Systems Inc., San Jose, Calif.) with PBS containing 0.1% Tween 20 and incubated for an additional one hour with a biotin labeled anti-IL-2 antibody (Endogen, M600B) and horse radish peroxidase coupled to strepavidin (PharMingen, #13047E). The plate was then washed again with 0.1% Tween 20/PBS, and an HRP-luminescent substrate was added to each well. Light intensity was then measured using a plate luminometer.

The percent inhibition (% I) for each well was calculated using the following formula:

% I=[(avg. untreated wells−treated well)/(avg. untreated wells)]×100

The average untreated well value (avg. untreated wells) is the arithmetic mean of 30 wells from the same assay plate treated with vehicle alone. Negative inhibition values result from local variations in the treated wells as compared to the untreated wells.

Mixtures are prepared and evaluated a number of times to provide a measure of the accuracy of the experiments. FIG. 14 provides illustrations of the results of a single representative experiment, with error bars and ranges being the result of data collected from various similarly performed experiments. The measured values of percent inhibition of IL-2 secretion by the agents alone and in combination, from conversion of raw data, are presented in Table 1 for the single representative experiment.

TABLE 1
Inhibition

| Chlorpromazine | Cyclosporine A (μM) | | | | | | | | |
|---|---|---|---|---|---|---|---|---|---|
| (μM) | 0 | 0.0077 | 0.015 | 0.031 | 0.062 | 0.12 | 0.25 | 0.5 | 0.99 |
| 0 | −14.1 | −11.7 | 0.35 | 28.8 | 55.6 | 74.0 | 78.6 | 80.1 | 82.3 |
| 0.6 | −13.3 | −11.1 | −4.7 | 33.6 | 54.8 | 67.2 | 78.7 | 84.9 | 84.2 |
| 1.2 | −18.7 | −10.8 | 4.6 | 28.0 | 57.8 | 73.4 | 78.0 | 81.9 | 83.2 |
| 2.5 | −12.7 | −14.8 | −8.7 | 25.0 | 55.6 | 76.1 | 81.2 | 82.1 | 85.8 |
| 5.0 | −13.7 | −5.9 | 6.7 | 36.1 | 66.1 | 77.4 | 81.3 | 85.7 | 86.8 |
| 9.9 | −1.9 | 9.5 | 25.9 | 58.8 | 76.7 | 85.0 | 87.9 | 88.4 | 88.1 |
| 20.0 | 24.7 | 49.6 | 67.4 | 84.0 | 89.2 | 92.0 | 91.5 | 93.3 | 89.8 |
| 40.0 | 80.7 | 86.9 | 89.4 | 94.4 | 94.8 | 94.8 | 95.3 | 94.7 | 94.3 |
| 80.0 | 94.70 | 92.1 | 94.9 | 89.3 | 95.8 | 92.7 | 93.3 | 94.9 | 94.3 |

Graphs 1410 and 1420 depict the individual responses of chlorpromazine and cyclosporine A, respectively, in suppressing the secretion of IL-2. Specific values 1411, 1421 are indicated by points, with the curves 1412, 1422 interpolating the points using a sinusoidal function. The 80% line 1413 represents the level of 80% inhibition.

The mean inhibitions from Table 1 are graphically depicted by the matrix of numbers in 1430, each number in a box representing the measured inhibition at a location of the 9×9 matrix corresponding to the relative position of the box. The concentrations of cyclosporine A increase according to the scale at the bottom of 1430, 1440, 1460, 1470 as locations move from left to right. Similarly, the concentrations of chlorpromazine increase according to the scale at the bottom of 1430, 1440, 1460, 1470 as locations move from bottom to top. The lines 1431 represents the interpolated graph of concentrations of the mixture that produce 80% inhibition, according to the measured data. The line 1432 represents the graph of concentrations of the mixture that produce 80% inhibition according to the Loewe Additivity Model. Matrix 1440 represents the standard error, or the standard deviation, associated with each location of the 9×9 assay array based on separate experiments which repeat the testing conditions, each number representing the standard error associated with the number's corresponding location.

Matrices 1460 and 1470 represent the difference between the measured inhibitions and calculated inhibitions based on the highest single agent and Bliss Independence Model, respectively, each number representing a difference between the measure inhibition and a model in the number's corresponding location in the 9×9 assay array. In general, larger numbers indicate greater synergy of the specific corresponding mixture. The Max=### 1461, 1471 shows the maximum difference achieved between a measured inhibition and a model's predicted inhibition for the corresponding matrix. The Sum (>0)=### 1462, 1472 shows the sum of all difference values in the corresponding matrix with difference values greater than zero; this may serve as a measure of the synergy of the combinations tested by the 9×9 array. The ± value with each Sum is the standard error associated with the difference value based on separate experiments which repeat the testing conditions.

Graph 1450 presents an isobologram of specific mixtures of chlorpromazine and cyclosporine A that are associated with a level of inhibition of 80%. Line 1451 represents the locus of concentrations that are expected to produce 80% inhibition, the line being interpolated based on the measured data. Line 1452 presents the locus of concentrations expected to produce an 80% inhibition based on the Loewe Additivity Model. The fact that line 1451 lies below line 1452 indicates the mixtures have synergistic inhibitory activity relative to what is expected from Loewe Additivity. The lines 1453 associated with each point of line 1451 represent the standard error associated with each point based on separate experiments which repeat the testing conditions. The Area 1454 represents the ratio of the area between lines 1451 and 1452 to the area between the line 1452 and the dotted lines 1456; this number also provides a measure of the synergy of all the combinations tested. The FIC80 1455 is the minimum value of the combination index for a point lying on 1451 yielding a fractional inhibitory concentration for 80% inhibition, which is represented by point 1457 with concentrations of cyclosporine A and chlorpromazine given by the X=### and Y=###, respectively. The combination index for 80% inhibition, CI80, is defined by

$$CI80 = \frac{{C}_{A}}{{C}_{A} \vert {I}_{A} = 0.80} + \frac{{C}_{B}}{{C}_{B} \vert {I}_{B} = 0.80}$$

where Cᵢ|Iᵢ=0.80 is the concentration of entity i such that the inhibition of the single entity i is equal to the value 0.80. In general, the lower the CI80 value the greater the synergy of the combination in producing 80% inhibition. The ± values again represent standard errors with the corresponding numbers based on separate experiments which repeat the testing conditions.

### Example 2

### Assay for Antiproliferative Activity of Compounds of Interest Against Non-Small Cell Lung Carcinoma A549

A total of 36 individual candidate entities were tested in 216 combinations for antiproliferative activity against non-small cell lung carcinoma A549. Following FIGS. 3 and 6, two constituent arrays 310, 320, 610, 620 holding various combinations of the candidate entities are created on plates with wells. “Aliquots” from corresponding wells of the constituent arrays are combined in the corresponding wells of a new plate to create a dilution array 330, 630 each well holding the candidate composition. Aliquots from wells of the dilution array 330, 630 are transferred to the corresponding wells of plates 340 holding an evaluative composition for the anti-proliferation assay, creating an assay array. The activity in wells of the assay array is then evaluated by looking for a fluorescence intensity signature indicative of antiproliferative activity.

Preparation of Compounds

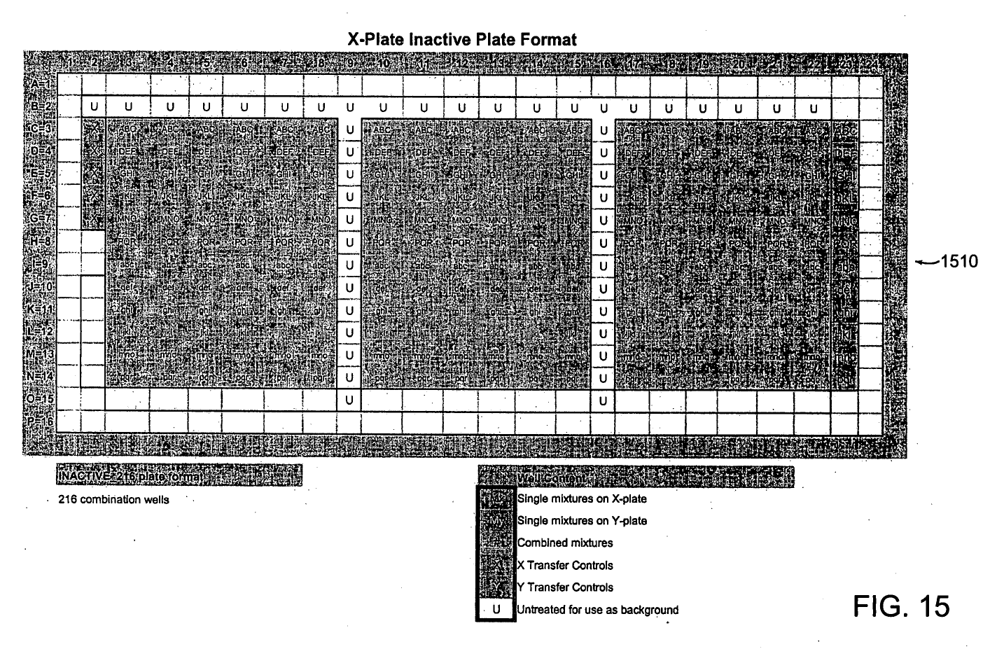

Stock solutions (1000×) of each candidate entity are prepared in DMSO. As shown in FIGS. 15 and 16, constituent arrays 1510 and 1610 holding two-fold serial dilutions of combinations of candidate entities, with respect to the stock solution concentrations, are assembled on 384-well plates, the concentration of any particular entity in a well location being substantially the same as the concentration of the particular entity in any other well containing the entity. One constituent array 1510 is configured as an X array, wherein each of a plurality of wells in each row contains the same composition. The other constituent array 1610 is configured as a Y array, wherein each of a plurality of wells in each column contains the same composition. Each constituent array 1510, 1610 is assembled such that at least one instance of each candidate entity is present in a composition of the array. Also, each entity used in a particular composition for a set of wells a constituent array 1510, 1610 is not utilized with any other entity of the particular composition in any other composition in any other constituent array 1510, 1610.

As shown in FIG. 17, a dilution array 1710 of candidate compositions is generated from the plates constituting the constituent arrays by combining aliquots from the corresponding wells of the constituent arrays into a corresponding well of the dilution array. Each combination of the dilution array is diluted into RPMI 1640 medium supplemented with 10% FBS, 2 mM glutamine, 1% penicillin, and 1% streptomycin. The dilution array contains three blocks of 6×12 wells, the combined wells of the three blocks having candidate compositions that contain all the candidate entities. The final concentrations of the candidate entities in the dilution array 1710 are ten times greater than used in the final assay array.

Tumor Cell Culture

Non-small cells lung carcinoma A549 (ATCC# CCL-185) cells are grown at 37±0.5° C. and 5% CO₂ in RPMI 1640 medium supplemented with 10% FBS, 2 mM glutamine, 1% penicillin, and 1% streptomycin.

Anti-Proliferation Assay

The anti-proliferation assay arrays are configured as a 384 well plates. The tumor cells were liberated from the culture flask using a solution of 0.25% trypsin. Cells are diluted in culture media such that 3000 cells are delivered in 20 μl of media into each assay array well. Assay plates are incubated for 16-24 hours at 37° C.±0.5° C. with 5% CO₂. Then, 6.6 μl of 10× stock solutions from the dilution array 1710 are added to corresponding wells of each assay plate with 40 μl of culture media to create an assay array. Assay plates are further incubated for 72 hours at 37° C.±0.5° C. Twenty-five microliters of 20% Alamar Blue in culture media warmed to 37° C.±0.5° C., is added to each assay well following the incubation period. Alamar Blue metabolism is quantified by the amount of fluorescence intensity 3.5-5.0 hours after addition. Quantification, using the LJL Analyst AD reader (LJL Biosystems, Sunnyvale, Calif.), is taken in the middle of the well with high attenuation, a 100 msec read time, an excitation filter at 530 nm, and an emission filter at 575 nm. Measurements are taken at the top of the well with stabilized energy lamp control; a 100 msec read time, an excitation filter at 530 nm, and an emission filter at 590 nm.

The percent inhibition (% I) for each well is calculated using the following formula:

% I=[(avg. untreated wells−treated well)/(avg. untreated wells)]×100

The average untreated well value (avg. untreated wells) is the arithmetic mean of 30 wells from the same assay plate treated with vehicle alone.

What is claimed is:

1. A method for evaluating an activity of each member of a set of combined compositions, each member of the set being a combination of a common plurality of constituent compositions, the method comprising:
providing, for each constituent composition, a constituent array of locations, each location associated with a specific concentration of such constituent composition, the arrays having a number corresponding to the plurality of constituent compositions;
providing an assay array of locations, each location of the assay array corresponding to a member of the set and being associated with a designated aliquot from each of the constituent arrays, wherein each aliquot is one of zero and non-zero; and
evaluating the activity of the combined composition at each location of the assay array.

2. A method according to claim 1, wherein at least one constituent composition includes an entity approved by a governmental regulatory agency for administration to a patient.

3. A method according to claim 1, wherein each of at least two constituent compositions include an entity approved by a governmental regulatory agency for administration to a patient.

4. A method according to claim 2, wherein the entity also has at least one of an established safety profile, a recognized pharmacology profile, and a recognized toxicity profile.

5. A method according to claim 1, wherein a plurality of locations of the assay array contain an evaluative composition pertinent to evaluating the activity of the combined composition.

6. A method according to claim 1, wherein the evaluative composition includes at least one test entity.

7. A method according to claim 1, wherein a particular concentration of at least one constituent composition in the assay array is designated based upon activity data of the at least one constituent composition.

8. A method according to claim 7, wherein the particular concentration corresponds approximately with a designated activity of the at least one constituent composition in the assay array.

9. A method according to claim 7 further comprising
evaluating an activity of the at least one constituent composition before providing its constituent array of locations;
wherein the activity data is based upon the evaluated activity of the at least one constituent composition before providing its constituent array of locations.

10. A method according to claim 7, wherein the activity data is based upon known activity data of the at least one constituent composition.

11. A method according to claim 7, wherein the activity data is in the form of at least one value of inhibition.

12. A method according to claim 7, wherein a plurality of particular concentrations of the at least one constituent composition in the assay array are based upon the activity data of the at least one constituent composition.

13. A method according to claim 12, wherein the plurality of particular concentrations correspond approximately with designated values of activity of the at least one constituent composition based upon the activity data of the at least one constituent composition.

14. A method according to claim 13, wherein the designated values of activity correspond to values of inhibition.

15. A method according to claim 14, wherein the designated values of inhibition are approximately between 20% and 80% of a maximum inhibition of the at least one constituent composition.

16. A method according to claim 12, wherein the plurality of particular concentrations include at least one concentration corresponding approximately to a selected value of activity of the at least one constituent composition based upon the activity data of the at least one constituent composition, and at least one other particular concentration based upon the selected value of activity.

17. A method according to claim 16, wherein the at least one other particular concentration is based upon a product of the selected concentration and a predetermined multiplicative factor.

18. A method according to claim 17, wherein the selected value of activity is a value of inhibition of 80% of a maximum inhibition of the at least one constituent composition, and the at least one specific concentration corresponds to approximately a two-fold multiple dilution from a concentration corresponding to the value of inhibition of 80% of the maximum inhibition of the at least one constituent composition.

19. A method according to claim 1, wherein at least one constituent array includes a series of members having successively greater dilutions of such constituent composition.

20. A method according to claim 19, wherein the successively greater dilutions encompass a total range of a factor of at least approximately 50,000, achieved in steps of a factor of at least approximately 3.

21. A method according to claim 19, wherein the successively greater dilutions encompass a total range of a factor of at least approximately 1,000, achieved in steps of a factor of at least approximately 4.

22. A method according to claim 19, wherein the successively greater dilutions encompass a total range of a factor of at least approximately 250, achieved in steps of a factor of at least approximately 2.

23. A method according to claim 1, wherein each of a plurality of locations of any constituent array has at least one corresponding location in any of the other constituent arrays, and the designated aliquot from each of the constituent arrays is taken from corresponding locations of the constituent arrays.

24. A method according to claim 23, wherein each constituent array includes at least one constituent composition with varying concentration in a plurality of locations, and wherein at least one concentration of the at least one constituent composition of one particular constituent array is not combined with every concentration of another constituent composition associated with another constituent array in the assay array.

25. A method according to claim 24, wherein the constituent array is embodied on more than one physical object, and the assay array is embodied on more than one physical object.

26. A method according to claim 25, wherein locations of any constituent array containing a particular concentration of the at least one entity are only present on one physical object.

27. A method according to claim 23, wherein all arrays have a common number of locations in corresponding positions of their respective physical objects.

28. A method according to claim 27, wherein each array is embodied in at least one plate.

29. A method according to claim 28, wherein each location of each array is realized by a well.

30. A method according to claim 1, wherein providing a constituent array of locations further comprises:
providing an origin set of unique locations in each constituent array, each location associated with a quantity of constituent composition associated with such array; and
providing, for each location of the origin set, a derivative set of unique locations in each constituent array, each location of a specific derivative set having a portion of constituent composition obtained from a location of the origin set.

31. A method according to claim 30, wherein the origin set of unique locations are embodied on a single physical object.

32. A method according to claim 30, wherein each location of any constituent array has a corresponding location in any of the other constituent arrays, and a plurality of locations, from any particular origin set location and its corresponding derivative set of locations of a given constituent array, are distinct from any locations of such constituent array that correspond to locations of an origin set location and its corresponding derivative set in any other constituent array.

33. A method according to claim 32, wherein a plurality of locations of at least one derivative set contains diluent.

34. A method according to claim 32, wherein, for at least one constituent array, each location of any derivative set contains at least one entity, all locations of a particular derivative set in the at least one constituent array containing substantially the same concentration of constituent composition.

35. A method according to claim 34, wherein each of a first and a second constituent array have an identically configured predetermined number of locations, each derivative set of the first constituent array arranged as a row of locations, and each derivative set of the second constituent array arranged as a column of locations.

36. A method according to claim 34, wherein each entity in a given derivative set of one constituent array is present in another derivative set of every other constituent array.

37. A method according to claim 36, wherein, for all constituent arrays, a combination of entities is only present in one derivative set.

38. A method according to claim 37, wherein each entity in the combination is not present with any other entity of the combination in any other location of any other constituent array.

39. A method according to claim 1, wherein each location of any constituent array has a corresponding location in any of the other constituent arrays, wherein the method further comprises:
providing, for each constituent array, a composition control in each location of a composition control set of such array,
wherein the composition control set of each constituent array is disposed so that all locations of the composition control set of a given constituent array are distinct from any locations of such constituent array that correspond to locations of the composition control set in any other constituent array.

40. A method according to claim 39, wherein at least one of the composition controls is a positive control and at least one of the composition controls is a negative control.

41. A method according to claim 40, wherein evaluating the activity of the combined composition at each location of the assay array further comprises:
providing a standard deviation value and an average value for each set of positive control locations and negative control locations of the composition control set of each physically distinct object of the assay array based upon values associated with an activity for each location of the respective set; and
providing a z-factor for each physically distinct object of the assay array based upon the standard deviation values and the average values.

42. A method according to claim 41, wherein the average values are embodied as numerical average values.

43. A method according to claim 41, wherein the average values are embodied as median values.

44. A method according to claim 40, wherein evaluating the activity of the combined composition at each location of the assay array further comprises:
providing a plurality of local quantized c-values, determined for at least one constituent composition of one composition control set of a physically distinct object of the assay array, the local quantized c-value being based upon a fractional value of activity, the fractional value of activity being a value of activity at a location of the one composition control set relative to a normalization value; and
providing a global c-value for each physically distinct object of the assay array based upon a numerical average of the plurality of local quantized c-values for each location of the physically distinct object of the composition control set.

45. A method according to claim 44, wherein the normalization value is associated with an expected activity level of zero.

46. A method according to claim 44, wherein the normalization value is associated with a background activity measurement.

47. A method according to claim 44, wherein the normalization value is a selected value.

48. A method according to claim 1, wherein each location of any constituent array has a corresponding location in any of the other constituent arrays, wherein the method further comprises:
providing an assay control in each location of an assay control set of the assay array,
wherein each location of the assay control set has a corresponding location in each constituent array.

49. A method according to claim 48, wherein the assay control set of one physical entity of the assay array has a plurality of locations which are adjacent to an edge of the physical entity.

50. A method according to claim 48, wherein the assay control set associated with one physical entity of the assay array has a plurality of wells which are arranged from one end of the physical entity to another end of the physical entity.

51. A method according to claim 48 further comprises
providing the assay control in at least one corresponding location of a constituent array before providing the assay array.

52. A method according to claim 48, wherein evaluating the activity of the combined composition includes:
evaluating a measured activity of the assay control in each location of the assay control set;
providing a deviation activity value for a plurality of locations of the assay array based upon the measured activity and an expected activity in one or more locations of the assay control set; and
assigning a corrected activity value for each of the plurality of locations of the assay array based upon the deviation activity values.

53. A method according to claim 52, wherein each of the plurality of locations of the assay array has the same expected value of activity.

54. A method according to claim 52, wherein providing the deviation value includes providing interpolated values based upon the measured activity in one or more locations of the assay control set.

55. A method according to claim 1, wherein evaluating the activity of the combined composition includes:
identifying erroneous activity values in one or more locations of the assay array; and
assigning a replacement value of activity in each location associated with the erroneous activity value.

56. A method according to claim 55, wherein the replacement value is assigned based upon the evaluated activity in one or more adjacent locations relative to the location associated with the erroneous activity value.

57. A method according to claim 55, wherein the replacement value is assigned based upon the concentration of at least one constituent composition in one or more adjacent locations relative to the location associated with the erroneous activity value.

58. A method according to claim 35, the method further comprising:
providing, for the assay array and each constituent array, a composition control in each location of a composition control set of such array, and an assay control in each location of an assay control set of such array,
wherein the composition control set of each array is disposed so that all locations of the composition control set of a particular array are distinct from any locations of such array that correspond to locations of the composition control set in any other array, and wherein the assay control set of each array is disposed so that each location of the assay control set of such array corresponds to a location of the assay control set in any other array.

59. A method according to claim 58, wherein providing an assay array further comprises:
providing a dilution array of locations, each location of the dilution array corresponding to a particular member of the set and being associated with a designated aliquot from each of the constituent arrays, wherein each aliquot is one of zero and non-zero, and
deriving the assay array of locations from the dilution array.

60. A method according to claim 59, wherein a concentration of a particular entity in a location of the dilution array is at least approximately one order of magnitude more dilute than the concentration of the particular entity in a designated constituent array.

61. A method according to claim 59, wherein a plurality of locations of the assay array contain an evaluative composition pertinent to evaluating the activity of the combined composition.

62. A method according to claim 59, wherein a concentration of a particular entity in a location of the assay array is at least approximately one order of magnitude more dilute than the concentration of the particular entity in a designated dilution array.

63. A method according to claim 59, wherein the assay array is embodied in a plurality of distinct physical objects.

64. A method according to claim 59, wherein each constituent array is embodied in at least one distinct physical object.

65. A method according to claim 59, wherein each location of the dilution array has a corresponding location in any of the constituent arrays, and the designated aliquot from each of the constituent arrays is taken from corresponding locations of the constituent arrays.

66. A method according to claim 65, wherein the arrays are embodied in physically distinct objects and all arrays have a common number of locations in corresponding positions of their respective physical objects.

67. A method according to claim 66, wherein each array is embodied in at least one plate.

68. A method according to claim 67, wherein each location of each array is realized by a well.

69. A method according to claim 68, wherein each constituent array includes a series of wells having successively greater dilutions of such constituent composition.

70. A method according to claim 69, wherein the successively greater dilutions encompass a total range of a factor of at least approximately 50,000, achieved in steps of a factor of at least approximately 3.

71. A method according to claim 69, wherein the successively greater dilutions encompass a total range of a factor of at least approximately 1,000, achieved in steps of a factor of at least approximately 4.

72. A method according to claim 69, wherein the successively greater dilutions encompass a total range of a factor of at least approximately 250, achieved in steps of a factor of at least approximately 2.

73. A method according to claim 68, wherein each constituent array includes at least one constituent composition with varying concentration in a plurality of locations, and wherein at least one concentration of the at least one constituent composition of one particular constituent array is not combined with every concentration of another constituent composition associated with another constituent array in the assay array.

74. A method according to claim 68, wherein a particular concentration of at least one constituent composition in the assay array corresponds approximately with a designated activity of the at least one constituent composition at the particular concentration.

75. A method according to claim 74, wherein a plurality of particular concentrations of the at least one constituent composition in the assay array correspond approximately with designated values of inhibition of the at least one constituent composition based upon the activity data of the at least one constituent composition, the designated values of inhibition being approximately between 20% and 80% of a maximum inhibition of the at least one constituent composition.

76. A method according to claim 58, wherein providing the constituent array includes providing the origin set and the derivative set on distinct physical objects.

77. A method according to claim 76, wherein a plurality of locations of the assay array contain an evaluative composition pertinent to evaluating the activity of the combined composition.

78. A method according to claim 76, wherein the assay array is embodied in a plurality of distinct physical objects.

79. A method according to claim 76, wherein the designated aliquot from each of the constituent arrays is taken from corresponding locations of the constituent arrays.

80. A method according to claim 79, wherein the arrays are embodied in physically distinct objects and all arrays have a common number of locations in corresponding positions of their respective physical objects.

81. A method according to claim 80, wherein each array is embodied in at least one plate.

82. A method according to claim 81, wherein each location of each array is realized by a well.

83. A method according to claim 82, wherein each constituent array includes a series of wells having successively greater dilutions of such constituent composition.

84. A method according to claim 83, wherein the successively greater dilutions encompass a total range of a factor of at least approximately 50,000, achieved in steps of a factor of at least approximately 3.

85. A method according to claim 83, wherein the successively greater dilutions encompass a total range of a factor of at least approximately 1,000, achieved in steps of a factor of at least approximately 4.

86. A method according to claim 83, wherein the successively greater dilutions encompass a total range of a factor of at least approximately 250, achieved in steps of a factor of at least approximately 2.

87. A method according to claim 82, wherein each constituent array includes at least one constituent composition with varying concentration in a plurality of locations, and wherein at least one concentration of the at least one constituent composition of one particular constituent array is not combined with every concentration of another constituent composition associated with another constituent array in the assay array.

88. A method according to claim 82, wherein a particular concentration of at least one constituent composition in the assay array corresponds approximately with a designated activity of the at least one constituent composition at the particular concentration.

89. A method according to claim 88, wherein a plurality of particular concentrations of the at least one constituent composition in the assay array correspond approximately with designated values of inhibition of the at least one constituent composition based upon the activity data of the at least one constituent composition, the designated values of inhibition being approximately between 20% and 80% of a maximum inhibition of the at least one constituent composition.

90. A method according to claim 1, wherein evaluating the activity of each member of the set of combined compositions includes providing a measure of synergy for a plurality of members of the set, the measure of synergy depending upon a measured value and a predicted value for each location of the set, each measured value being pertinent to the activity in one location of the set, and each predicted value being calculated from a model.

91. A method according to claim 90, wherein the model depends upon measured values pertinent to an activity of at least one entity of a candidate composition in the one location of the set.

92. A method according to claim 91, wherein the predicted value is the activity of the at least one entity of the candidate composition.

93. A method according to claim 91, wherein the predicted value is calculated from a Bliss Independence Model.

94. A method according to claim 91, wherein the predicted value is calculated from a Loewe Additivity Model.

95. A method according to claim 90, wherein the measure of synergy is a difference between a measured value and a predicted value for each location of the set.

96. A method according to claim 95, wherein the measure of synergy is a sum of the difference between the measured value and predicted value for a plurality of locations of the set.

97. A method according to claim 95, wherein the measure of synergy is a representation of the concentrations of entities in a candidate composition associated with a specific level of activity derived from interpolation of a plurality of measured values.

98. A method according to claim 95, wherein evaluating the activity includes replacing particular measured values with calculated values that maintain a smooth monotonically changing surface of values with respect to each calculated value and measured values at locations adjacent to the calculated value.

99. A method of evaluating the activity of a set of compositions in an array, the method comprising:
determining a measured value for each location of a set of compositions, for each of a plurality of sets of the array, pertinent to the activity thereof, wherein each set of the array includes substantially the same set of compositions arranged in corresponding locations;
for each of the locations of the sets of the array, determining predicted values of activity according to each of a plurality of models; and
determining the activity of the set of compositions based upon the measured values and predicted values using at least one statistical method.

100. A method according to claim 99, wherein determining the activity includes determining the activity based upon the difference between the measured value and the predicted value in corresponding locations of each set for each of the plurality of models.

101. A method according to claim 100, wherein determining the activity includes providing a summation of all difference values exceeding a difference threshold for each set of the array.

102. A method according to claim 99, wherein using at least one statistical method includes determining a standard error of activity associated with a location of a set based upon the measured values in corresponding locations of each of the plurality of sets of the array.

103. A method according to claim 102, wherein determining the activity of the set includes determining a measure of error of the activity of the set based upon the standard error of activity associated with a plurality of locations of the set.

104. A method according to claim 103, wherein determining the measure of error includes determining a square-root of the sum of the squares of the standard errors of activity of the plurality of locations.

105. A method according to claim 99, wherein using at least one statistical method includes determining an average measured value associated with a location of a set based upon the measured values in corresponding locations of each of the plurality of sets of the array.

106. A method according to claim 99, wherein using at least one statistical method includes determining a ratio of an average measured value to a standard error associated with a location of a set based upon the measured values in corresponding locations of each of the plurality of sets of the array.

107. A method according to claim 99, wherein the measured values and predicted values are expressed in terms of inhibition.

108. An assay array having a set of combined compositions, each member of the set being a combination of a common plurality of constituent compositions, the assay array comprising:
an array of locations, each location corresponding to a member of the set and being associated with a designated aliquot from each of a plurality of constituent arrays, each constituent array having locations holding a specific concentration of a constituent composition, the constituent arrays having a number corresponding to the plurality of constituent compositions, each aliquot is one of zero and non-zero.

109. An assay array having a set of combined compositions, each member of the set being a combination of a common plurality of constituent compositions, the assay array comprising:
an array of locations, each location corresponding to a member of the set and being associated with a designated aliquot from of a specific concentration of a constituent composition, each aliquot is one of zero and non-zero,
wherein a particular concentration of at least one constituent composition in the assay array is designated based upon activity data of the at least one constituent composition.

110. An assay array according to claim 109, wherein the particular concentration corresponds approximately with a designated activity of the at least one constituent composition in the assay array.

111. An assay array according to claim 109, wherein the activity data is based upon known activity data of the at least one constituent composition.

112. An assay array according to claim 109, wherein a plurality of particular concentrations of the at least one constituent composition in the assay array are based upon the activity data of the at least one constituent composition.

113. An assay array according to claim 112, wherein the plurality of particular concentrations correspond approximately with designated values of activity of the at least one constituent composition based upon the activity data of the at least one constituent composition.

114. An assay array according to claim 113, wherein the designated values of activity correspond to values of inhibition.

115. An assay array according to claim 114, wherein the designated values of inhibition are approximately between 20% and 80% of a maximum inhibition of the at least one constituent composition.

116. An assay array according to claim 112, wherein the plurality of particular concentrations include at least one concentration corresponding approximately to a selected value of activity of the at least one constituent composition based upon the activity data of the at least one constituent composition, and at least one other particular concentration based upon the selected value of activity.

117. An assay array according to claim 116, wherein the at least one other particular concentration is based upon a product of the selected concentration and a predetermined multiplicative factor.

118. An assay array according to claim 117, wherein the selected value of activity is a value of inhibition of 80% of a maximum inhibition of the at least one constituent composition, and the at least one specific concentration corresponds to approximately a two-fold multiple dilution from a concentration corresponding to the value of inhibition of 80% of the maximum inhibition of the at least one constituent composition.

119. An assay array having a set of combined compositions, each member of the set being a combination of a common plurality of constituent compositions, the assay array comprising:
an array of locations, each location corresponding to a member of the set and being associated with a designated aliquot from of a specific concentration of a constituent composition, each aliquot is one of zero and non-zero,
wherein at least one concentration of a particular constituent composition in the assay array is not combined with every concentration of a different constituent composition in the assay array.

120. An assay array according to claim 119, wherein the assay array is embodied on more than one physical object.

121. A plurality of arrays for evaluating an activity of each member of a set of combined compositions, each member of the set being a combination of a common plurality of constituent compositions, the plurality of arrays comprising:
for each constituent composition, a constituent array of locations, each location associated with a specific concentration of such constituent composition, the constituent arrays having a number corresponding to the plurality of constituent compositions, each location of any constituent array having a corresponding location in any of the other constituent arrays;
an assay array of locations, each location of the assay array corresponding to a member of the set and being associated with a designated aliquot from each of the constituent arrays, each aliquot is one of zero and non-zero; and
an assay control in each location of an assay control set of the assay array,
wherein each location of the assay control set has a corresponding location in each constituent array.

122. A plurality of arrays according to claim 121, wherein the assay control set of one physical entity of the assay array has a plurality of locations which are adjacent to an edge of the physical entity.

123. A plurality of arrays according to claim 121, wherein the assay control set associated with one physical entity of the assay array has a plurality of wells which are arranged from one end of the physical entity to another end of the physical entity.

124. A plurality of arrays according to claim 121, wherein the assay control is in at least one corresponding location of a constituent array before being in the assay array.

125. A plurality of constituent arrays for producing an assay array, each constituent array comprising:
an array of locations for holding a constituent composition, each location associated with a specific concentration of such constituent composition, the constituent arrays having a number corresponding to the plurality of constituent compositions, each constituent array including:
(i) an origin set of unique locations, each origin set location associated with a quantity of constituent composition associated with such array; and
(ii) for each location of the origin set, a derivative set of unique locations, each location of a specific derivative set having a portion of constituent composition obtained from a location of the origin set.

126. A plurality of constituent arrays according to claim 125, wherein the origin set of unique locations are embodied on a single physical object.

127. A plurality of constituent arrays according to claim 125, wherein each location of any constituent array has a corresponding location in any of the other constituent arrays, and a plurality of locations, from any particular origin set location and its corresponding derivative set of locations of a given constituent array, are distinct from any locations of such constituent array that correspond to locations of an origin set location and its corresponding derivative set in any other constituent array.

128. A plurality of constituent arrays according to claim 127, wherein a plurality of locations of at least one derivative set contains diluent.

129. A plurality of constituent arrays according to claim 127, wherein, for at least one constituent array, each location of any derivative set contains at least one entity, all locations of a particular derivative set in the at least one constituent array containing substantially the same concentration of constituent composition.

130. A plurality of constituent arrays according to claim 129, wherein each of a first and a second constituent array have an identically configured predetermined number of locations, each derivative set of the first constituent array arranged as a row of locations, and each derivative set of the second constituent array arranged as a column of locations.

131. A plurality of constituent arrays according to claim 129, wherein each entity in a given derivative set of one constituent array is present in another derivative set of every other constituent array.

132. A plurality of constituent arrays according to claim 131, wherein, for all constituent arrays, a combination of entities is only present in one derivative set.

133. A plurality of constituent arrays according to claim 132, wherein each entity in the combination is not present with any other entity of the combination in any other location of any other constituent array.

134. A plurality of arrays for evaluating an activity of each member of a set of combined compositions, each member of the set being a combination of a common plurality of constituent compositions, the plurality of arrays comprising:
for each constituent composition, a constituent array of locations, each location associated with a specific concentration of such constituent composition, the constituent arrays having a number corresponding to the plurality of constituent compositions, each location of any constituent array having a corresponding location in any of the other constituent arrays;
an assay array of locations, each location of the assay array corresponding to a member of the set and being associated with a designated aliquot from each of the constituent arrays, each aliquot is one of zero and non-zero; and
a composition control in each location of a composition control set,
wherein the composition control set of each constituent array is disposed so that all locations of the composition control set of a given constituent array are distinct from any locations of such constituent array that correspond to locations of the composition control set in any other constituent array, the locations of all composition control sets having a corresponding location in the assay array.

135. A plurality of arrays according to claim 134, wherein at least one of the composition controls is a positive control and at least one of the composition controls is a negative control.

136. A plurality of arrays according to claim 134 further comprising:
an assay control in each location of an assay control set of the assay array,
wherein each location of the assay control set has a corresponding location in each constituent array.

137. A plurality of arrays according to claim 136, wherein a particular concentration of at least one constituent composition in the assay array is designated based upon activity data of the at least one constituent composition.

138. A plurality of arrays according to claim 137, wherein a plurality of particular concentrations of the at least one constituent composition in the assay array correspond approximately with designated values of inhibition of the at least one constituent composition based upon the activity data of the at least one constituent composition, the designated values of inhibition being approximately between 20% and 80% of a maximum inhibition of the at least one constituent composition.

139. A plurality of arrays according to claim 136, wherein the locations of combinations of the common plurality of constituent compositions of the assay array do not correspond to every virtual location of a virtual assay array representing combinations of the constituent compositions, the virtual assay array having a set of virtual locations, the set of virtual locations corresponding with every possible combination of specific concentrations of constituent compositions utilized in the assay array.

140. A plurality of arrays according to claim 138, wherein the locations of combinations of the common plurality of constituent compositions of the assay array do not correspond to every virtual location of a virtual assay array representing combinations of the constituent compositions, the virtual assay array having a set of virtual locations, the set of virtual locations corresponding with every possible combination of specific concentrations of constituent compositions utilized in the assay array.

141. A computer program product for use on a computer system for evaluating a combination effect in a plurality of locations of an assay array, the computer readable program code including:
(a) a module for collecting an evaluated activity in the plurality of locations of the assay array;
(b) program code for providing a measure value in the plurality of locations of the assay array, the measure values based upon the evaluated activity in the plurality of locations;
(c) program code for providing a predicted value for each of the plurality of locations of the assay array, the predicted values based upon a model; and
(d) program code for evaluating a combination effect for each of the plurality of locations of the assay array, the evaluation based upon the measured values and predicted values.

142. A computer program product according to claim 141, wherein the model depends upon measured values pertinent to an activity of at least one entity of a candidate composition in a location of the set.

143. A computer program product according to claim 142, wherein the predicted value is the activity of the at least one entity of the candidate composition.

144. A computer program product according to claim 142, wherein the predicted value is calculated from a Bliss Independence Model.

145. A computer program product according to claim 142, wherein the predicted value is calculated from a Loewe Additivity Model.

146. A computer program product according to claim 141, wherein the evaluation is a set of differences between a measured value and a predicted value for each of the plurality of locations.

147. A computer program product according to claim 146, wherein the evaluation is a sum of the difference between the measured value and predicted value for each of the plurality of locations.

148. A computer program product according to claim 146, wherein the evaluation is a representation of the concentrations of entities in a candidate composition associated with a specific level of activity derived from interpolation of the set of differences between the measure value and predicted value for each of the plurality of locations.

149. A computer program product according to claim 146 further comprising:
(e) program code for replacing particular measured values with calculated values such that a smooth monotonically changing activity surface is produced from the calculated values and measured values that are not replaced.

150. A computer program product according to claim 149, wherein the measured value is replaced by a corresponding calculated value when the difference between the measured value and the corresponding predicted value exceeds a given threshold value.

151. A computer program product according to claim 146, wherein a subset of the plurality of locations correspond to a plurality of locations containing an assay control, and the predicted values include an identical value corresponding to an expected activity associated with each location of the assay control, the computer program product further comprising:
(e) program code for providing correction values for to the plurality of locations based upon a set of differences between each measured value and predicted value in the assay control locations.
